(19) United States
(12) Patent Application Publication
(10) Pub. No.: US 2025/0334536 A1
(43) Pub. Date: Oct. 30, 2025

(54) MODIFIED OXIDOREDUCTASE AND ITS APPLICATION

(71) Applicant: National Yang Ming Chiao Tung University, Hsinchu City (TW)

(72) Inventors: Hsin-Chieh LIN, Hsinchu City (TW); Yuan-Hao HSIAO, Hsinchu City (TW); Rajan Deepan CHAKRAVARTHY, Hsinchu City (TW)

(21) Appl. No.: 19/189,301

(22) Filed: Apr. 25, 2025

Related U.S. Application Data
(60) Provisional application No. 63/638,945, filed on Apr. 26, 2024.

(51) Int. Cl. G01N 27/327; C12N 9/04; C12Q 1/00

(52) U.S. Cl. CPC G01N 27/3271; C12N 9/0006; C12Q 1/003; C12Y 101/03004

(57) ABSTRACT

A modified redox enzyme includes a dehydrogenated redox enzyme and a modification chain segment. The dehydrogenated redox enzyme includes at least one dehydrogenated thiol group, and the modification chain segment has a structure of Formula (1) and is bonded to the dehydrogenated thiol group. The dehydrogenated redox enzyme includes at least one dehydrogenated amino group, and the modification chain segment has the structure of Formula (2) and is bonded to the dehydrogenated amino group.

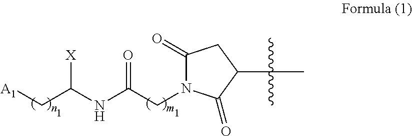

A₁ is a first chain segment having a first π-conjugated system, X is hydrogen, alkyl, carboxyl, amide, or ester group, n₁ is an integer from 0 to 20, and m₁ is an integer from 0 to 20;

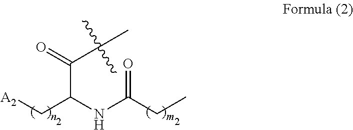

A₂ is a second chain segment having a second π-conjugated system, n₂ is an integer from 0 to 20, and m₂ is an integer from 0 to 20.

### CROSS-REFERENCE TO RELATED APPLICATION

This application claims priority to U.S. Provisional Application Ser. No. 63/638,945, filed Apr. 26, 2024, which is herein incorporated by reference.

### BACKGROUND

### Field of Invention

The present disclosure relates to a modified redox enzyme and its applications, particularly to a modified redox enzyme and its use in nanoparticle compositions, electrochemical sensors, nanomedicines, and nanomedicine complexes.

### Description of Related Art

In the prior art, redox enzymes have been widely used in various biomedical and industrial fields, such as functional components in drug delivery systems or active substances in sensors and catalytic reactions. However, natural enzymes are sensitive to environmental conditions such as temperature, pH, organic solvents, or interfacial tension, which can lead to loss of activity and limit their stability and reusability in practical applications. To address these issues, researchers have developed various nanozymes designed to mimic the structure and function of natural enzymes. Although nanozymes can effectively protect the enzyme's active center through their unique nanostructures, thereby enhancing stability and catalytic efficiency, they still have some drawbacks. For example, nanozymes lack the high specificity of natural enzymes, and certain nanozyme materials (such as metals or metal oxides) may raise biocompatibility concerns, increasing the risk of in vivo applications. Moreover, the synthesis of nanozymes often requires harsh conditions such as high temperature and pressure, resulting in complicated processes and increased production costs, which pose challenges for large-scale production and clinical translation. Therefore, it remains an urgent technical challenge to provide a solution that enhances enzyme stability and catalytic efficiency, reduces the risk of denaturation and inactivation, and enables the formation of multifunctional enzyme nanoparticles in combination with drugs.

### SUMMARY

According to one or more embodiments of the present disclosure, a modified oxidoreductase includes a dehydrogenated oxidoreductase and a modification chain segment. The dehydrogenated oxidoreductase has at least one dehydrogenated thiol group or at least one dehydrogenated amino group. When the dehydrogenated oxidoreductase has the at least one dehydrogenated thiol group, the modification chain segment includes a structure represented by Formula (1) and is bonded to the at least one dehydrogenated thiol group. When the dehydrogenated oxidoreductase has the at least one dehydrogenated amino group, the modification chain segment includes a structure represented by Formula (2) and is bonded to the at least one dehydrogenated amino group.

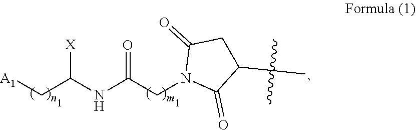

wherein A₁ is a first chain segment having a first π-conjugated system, X is hydrogen, alkyl, carboxyl, amide, or ester group, n₁ is an integer from 0 to 20, and m₁ is an integer from 0 to 20;

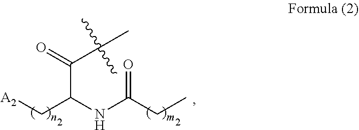

wherein A₂ is a second chain segment having a second π-conjugated system, n₂ is an integer from 0 to 20, and m₂ is an integer from 0 to 20.

In one or more embodiments of the present disclosure, the first π-conjugated system or the second π-conjugated system is a π-conjugated system within an aromatic ring.

In one or more embodiments of the present disclosure, the first chain segment and the second chain segment are each represented by Formula (3), Formula (4), or Formula (5):

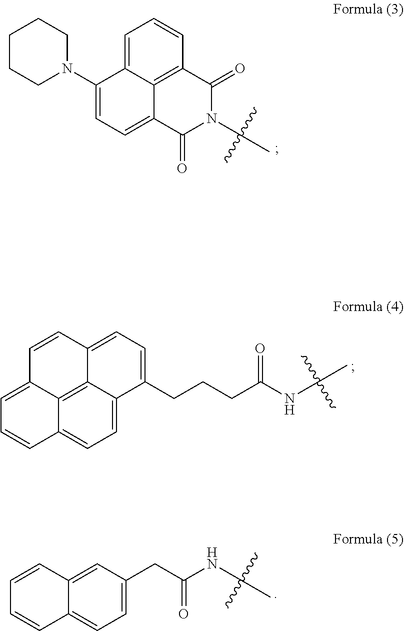 [SMILES: *N1C(=O)c2cccc3c(N4CCCCC4)ccc(c23)C1=O ; *NC(=O)CCCc1ccc2ccc3cccc4ccc1c2c34 ; *NC(=O)Cc1ccc2ccccc2c1]

In one or more embodiments of the present disclosure, the dehydrogenated oxidoreductase is obtained by dehydrogenation of glucose oxidase, glucose dehydrogenase, pyruvate oxidase, xanthine oxidase, acetylcholinesterase, lactate oxidase, urate oxidase, uricase, pyrroloquinoline quinone-dependent glucose dehydrogenase-A, pyrroloquinoline quinone-dependent glucose dehydrogenase-B, NAD(P)-dependent glutamate dehydrogenase, FAD-dependent glutamate dehydrogenase, cholesterol oxidase, or a sulfur-containing enzyme.

In one or more embodiments of the present disclosure, a number of the at least one dehydrogenated thiol group is from 1 to 20, or a number of the at least one dehydrogenated amino group is from 1 to 20.

According to one or more embodiments of the present disclosure, a preparation method of a nanoparticle composition includes: placing a plurality of the aforementioned modified oxidoreductases of claim 1 in an aldehyde or aldonic acid solution at a concentration of 0.1 mM to 0.31 mM.

In one or more embodiments of the present disclosure, the aldehyde or aldonic acid solution is a monoaldehyde solution, dialdehyde solution, aromatic aldehyde solution, α,β-unsaturated aldehyde solution, hydroxyaldehyde solution, ketoaldehyde solution, aldonic acid solution, or combinations thereof.

According to one or more embodiments of the present disclosure, a nanoparticle composition includes a plurality of enzyme nanoparticles, each of the enzyme nanoparticles being formed by crosslinking a plurality of modified oxidoreductases and a plurality of aldehyde or aldonic acid molecules, wherein each of the modified oxidoreductases includes a dehydrogenated oxidoreductase and a modification chain segment. The dehydrogenated oxidoreductase has at least one dehydrogenated thiol group or at least one dehydrogenated amino group. When the dehydrogenated oxidoreductase has the at least one dehydrogenated thiol group, the modification chain segment includes a structure represented by Formula (1) and is bonded to the at least one dehydrogenated thiol group. When the dehydrogenated oxidoreductase has the at least one dehydrogenated amino group, the modification chain segment includes a structure represented by Formula (2) and is bonded to the at least one dehydrogenated amino group.

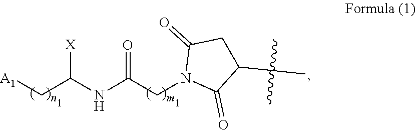

wherein A₁ is a first chain segment having a first π-conjugated system, X is hydrogen, alkyl, carboxyl, amide, or ester group, n₁ is an integer from 0 to 20, and m₁ is an integer from 0 to 20;

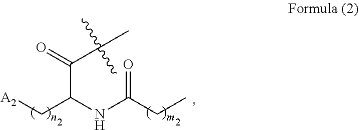

wherein A₂ is a second chain segment having a second π-conjugated system, n₂ is an integer from 0 to 20, and m₂ is an integer from 0 to 20.

In one or more embodiments of the present disclosure, an average diameter of the enzyme nanoparticles is from 10 nanometers to 5000 nanometers.

In one or more embodiments of the present disclosure, the aldehyde or aldonic acid molecules are monoaldehyde, dialdehyde, aromatic aldehyde, α,β-unsaturated aldehyde, hydroxyaldehyde, ketoaldehyde, aldonic acid, or combinations thereof.

In one or more embodiments of the present disclosure, the dehydrogenated oxidoreductase is obtained by dehydrogenation of glucose oxidase, and the nanoparticle composition has a catalytic efficiency (K_{cat}/K_{M}) for glucose greater than 0.5×10⁴ s⁻¹·M⁻¹ and less than 5.0×10⁴ s⁻¹·M⁻¹, wherein K_{cat} is the catalytic constant and K_{M} is the Michaelis constant.

According to one or more embodiments of the present disclosure, an electrical signal sensor includes an electrode layer and a sensing layer disposed on a surface of the electrode layer, wherein the sensing layer includes the aforementioned nanoparticle composition.

According to one or more embodiments of the present disclosure, a nanomedicine includes the aforementioned nanoparticle composition.

According to one or more embodiments of the present disclosure, a nanomedicine complex includes the aforementioned nanoparticle composition and a drug molecule bound in a releasable form to any of the modified oxidoreductases in the nanoparticle composition.

In one or more embodiments of the present disclosure, the drug molecule is an anticancer drug.

According to the aforementioned embodiments of the present disclosure, the modified redox enzyme exhibits amphiphilic molecular properties by grafting hydrophobic chain segments, enabling stable dispersion in aqueous solutions and spontaneous assembly into multifunctional enzyme nanoparticles. Such self-assembled structures help enhance the enzyme's conformational stability and functional performance, allowing it to maintain high activity and catalytic efficiency under various pH and temperature conditions, thereby effectively reducing loss due to denaturation or activity loss during application. Given their excellent stability and biocompatibility, these enzyme nanoparticles hold broad application potential in biomedical, environmental, food, and bioenergy fields.

### BRIEF DESCRIPTION OF THE DRAWINGS

To make the above and other objectives, features, advantages, and embodiments of the present disclosure more apparent and understandable, the accompanying drawings are described as follows:

FIG. 1A to FIG. 1I are scanning electron microscope (SEM) images of the modified glucose oxidase in glutaraldehyde solutions of different concentrations;

FIG. 2A to FIG. 2D are particle size distribution graphs of the enzyme nanoparticles shown in FIG. 1C to FIG. 1F, respectively;

FIG. 3A is a plot of the average particle size of the enzyme nanoparticles in FIG. 1A to FIG. 1H under different glutaraldehyde concentrations;

FIG. 3B is a plot of the reaction rate of the nanoparticle composition under different glutaraldehyde concentrations;

FIG. 4 is a current response plot of the nanoparticle composition under different glutaraldehyde and glucose concentrations;

FIG. 5A and FIG. 5B are UV-Vis absorption spectra of the nanoparticle composition and the modified glucose oxidase, respectively;

FIG. 6A and FIG. 6B are absorbance-time plots of the nanoparticle composition and the modified glucose oxidase at different glucose concentrations, respectively;

FIG. 6C is a calibration curve of ABTS⁺·absorbance versus concentration;

FIG. 6D and FIG. 6E are Michaelis-Menten plots of the nanoparticle composition and the modified glucose oxidase, respectively;

FIG. 6F and FIG. 6G are Lineweaver-Burk double reciprocal plots of the nanoparticle composition and the modified glucose oxidase, respectively;

FIG. 7 is a plot of the reaction rate of the nanoparticle composition under different pH values;

FIG. 8A is the UV-Vis absorption spectra of compound of Formula (C) at different concentrations and the modified glucose oxidase in dimethyl sulfoxide;

FIG. 8B is the concentration-absorbance calibration curve of compound of Formula (C);

FIG. 9A and FIG. 9B are the MALDI-MS spectra of the modified glucose oxidase and glucose oxidase, respectively;

FIG. 10A is a current density variation plot of the sensor over seven days in phosphate-buffered saline (PBS);

FIG. 10B is a bar chart showing the current density variation of the sensor after seven days in PBS;

FIG. 10C is a fluorescence intensity-concentration plot of the sensor in PBS;

FIG. 10D is a cumulative release efficiency plot of the sensor over seven days in PBS;

FIG. 11 is a current variation plot of the sensor upon the addition of different substrates;

FIG. 12 is a plot of the reaction rate of the nanoparticle composition at different temperatures;

FIG. 13A is a transmission electron microscope (TEM) image of the DOX-loaded nanomedicine complex;

FIG. 13B is a partially enlarged schematic of FIG. 13A; and

FIG. 14 is a side view schematic diagram of an electrochemical sensor according to some embodiments of the present disclosure.

### DETAILED DESCRIPTION

The present disclosure illustrates multiple embodiments with reference to the figures. For the sake of clarity, numerous practical details are provided in the following description. However, it should be understood that these practical details are not intended to limit the present disclosure. That is, in some embodiments of the present disclosure, such practical details are not necessary and should not be construed as limitations.

In the present disclosure, polymers or groups may sometimes be represented using skeleton formulas. This notation omits carbon atoms, hydrogen atoms, and carbon-hydrogen bonds. Of course, when atoms or atomic groups are explicitly shown in the structural formulas, the depiction shall prevail.

Unless otherwise specified, the numerical ranges mentioned in the present disclosure include their endpoints. For example, expressions such as “A to B,” “A-B,” or similar phrases are intended to mean A, B, or any value between them.

The present disclosure provides a modified redox enzyme that, after being grafted with hydrophobic chain segments, exhibits amphiphilic molecular properties, allowing it to remain stable in solution and further self-assemble into multifunctional enzyme nanoparticles. This self-assembled structure enhances the structural stability and functional performance of the enzyme, enabling the enzyme nanoparticles to maintain high activity and catalytic efficiency across a wide range of pH values and temperatures, thereby reducing enzyme loss caused by denaturation or inactivation. Due to their stability and biocompatibility, the enzyme nanoparticles are applicable in various fields, including biomedicine, environmental technology, the food industry, and biofuels. Specific applications include drug synthesis, biosensing, and disease diagnosis in the medical field; water treatment and pollutant degradation in environmental technology; lactose hydrolysis and starch saccharification in the food industry; and improving enzyme utilization efficiency in biofuel production.

It should first be understood that redox enzymes (e.g., glucose oxidase) are highly hydrophilic proteins. When placed in aqueous solution, they tend to dissolve readily and exist in a homogeneously dispersed state, lacking sufficient hydrophobic driving force for self-assembly. As a result, it is difficult for them to form stable nanoparticles with well-defined interfaces and structures. To address this, the modified redox enzyme disclosed herein is obtained by chemically modifying the redox enzyme to impart amphiphilic characteristics-namely, the presence of both hydrophilic and hydrophobic chain segments-thereby promoting its self-assembly into stable nanostructures in aqueous solution. According to structural differences, the modified redox enzymes of the present disclosure can be categorized into two types, both of which are capable of achieving the intended effects described herein, as explained below.

### <Modified Redox Enzyme>

The modified redox enzyme of the first type is obtained by grafting a hydrophobic modification chain segment to a redox enzyme through a thiol-maleimide Michael addition reaction. In this embodiment, the hydrophobic modification chain segment is bonded to the redox enzyme via a thioether bond. Specifically, the modified redox enzyme includes a dehydrogenated redox enzyme and a hydrophobic modification chain segment. The dehydrogenated redox enzyme includes at least one dehydrogenated thiol group, and the modification chain segment is bonded to the dehydrogenated thiol group. The modification chain segment has the structure of Formula (1):

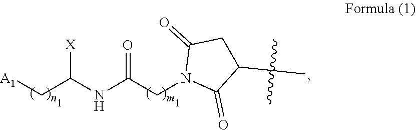

wherein A₁ is a first chain segment having a first π-conjugated system; X is hydrogen, an alkyl group (e.g., methyl, ethyl, propyl, butyl, pentyl, hexyl, heptyl, octyl, nonyl, decyl, isopropyl, isobutyl, sec-butyl, tert-butyl, isopentyl, neopentyl, sec-pentyl, tert-pentyl, dodecyl, tridecyl, tetradecyl, pentadecyl, hexadecyl, heptadecyl, octadecyl, nonadecyl, eicosyl, docosyl, 3-methylbutyl, or 2-methylbutyl), a carboxyl group, an amide group, or an ester group (e.g., methyl formate, ethyl formate, propyl formate, butyl formate, pentyl formate, hexyl formate, methyl acetate, ethyl acetate, propyl acetate, butyl acetate, isobutyl acetate, pentyl acetate, hexyl acetate, octyl acetate, methyl propionate, ethyl propionate, propyl propionate, butyl propionate, methyl butyrate, ethyl butyrate, propyl butyrate, ethyl hexanoate, ethyl heptanoate, ethyl octanoate, ethyl decanoate, methyl benzoate, ethyl benzoate, methyl salicylate, ethyl salicylate, or methyl p-hydroxybenzoate); n₁ is an integer from 0 to 20; and m₁ is an integer from 0 to 20.

The modified redox enzyme of the second type is prepared by grafting a hydrophobic modification chain segment onto the redox enzyme via a condensation reaction (amide bond formation reaction). In other words, in this embodiment, the hydrophobic modification chain segment is bonded to the redox enzyme through an amide bond. Specifically, the modified redox enzyme includes a dehydrogenated redox enzyme and a hydrophobic modification chain segment, wherein the dehydrogenated redox enzyme has at least one dehydrogenated (i.e., hydrogen-removed) amino group, and the modification chain segment is bonded to the dehydrogenated amino group. The modification chain segment has a structure of Formula (2):

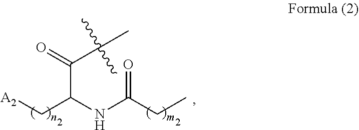

wherein A₂ is a second chain segment having a second π-conjugated system; n₂ is an integer from 0 to 20; and m₂ is an integer from 0 to 20.

In some embodiments, the first π-conjugated system and the second π-conjugated system may each be an aromatic ring π-conjugated system. That is, the π-conjugated system exists within the conjugated electron cloud of the aromatic ring, formed by the carbon atoms of the aromatic ring with alternating single and double bonds to create an extended conjugated structure. Specifically, the first chain segment and the second chain segment may each have the structure of Formula (3), Formula (4), or Formula (5):

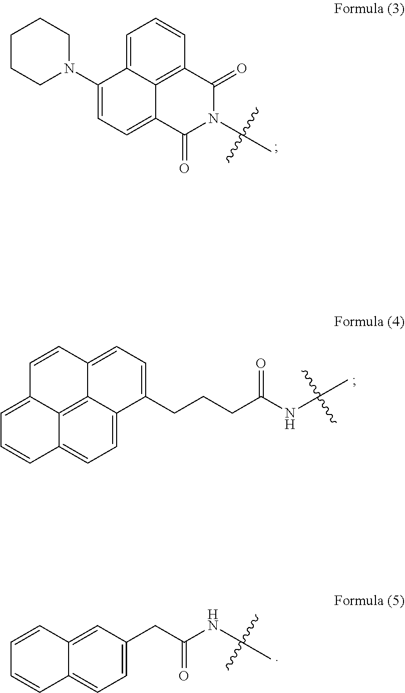 [SMILES: *N1C(=O)c2cccc3c(N4CCCCC4)ccc(c23)C1=O ; *NC(=O)CCCc1ccc2ccc3cccc4ccc1c2c34 ; *NC(=O)Cc1ccc2ccccc2c1]

In some preferred embodiment, the first chain segment and the second chain segment each have the structure of Formula (3). Since the aromatic ring possesses a stable resonance structure, it can impart good chemical stability to the modified chain segment, thereby reducing the risk of decomposition under various environmental conditions. Moreover, the hydrophobicity of the aromatic ring helps the modified redox enzyme form distinct hydrophilic and hydrophobic regions in aqueous solution, effectively promoting its self-assembly behavior, further stabilizing the formation of the nanostructure, and enhancing the overall stability and functional performance of the enzyme nanoparticles.

In some embodiments, the aforementioned dehydrogenated redox enzyme may be obtained by dehydrogenation of glucose oxidase, glucose dehydrogenase, pyruvate oxidase, catalase, xanthine oxidase, acetylcholinesterase, lactate oxidase, uricase, urate oxidase, pyrroloquinoline quinone glucose dehydrogenase-A, pyrroloquinoline quinone glucose dehydrogenase-B, NAD(P)-dependent glutamate dehydrogenase, FAD-dependent glutamate dehydrogenase, cholesterol oxidase, or sulfur-containing enzymes. In embodiments where the redox enzyme is glucose oxidase, glucose dehydrogenase, pyrroloquinoline quinone glucose dehydrogenase-A, or pyrroloquinoline quinone glucose dehydrogenase-B, the modified redox enzyme can catalyze redox reactions involving glucose. For example, glucose oxidase can catalyze the oxidation of glucose to glucono-δ-lactone and hydrogen peroxide (H₂O₂) in the presence of oxygen.

Regarding modified redox enzyme of the first type, in some embodiments, the number of dehydrogenated thiol groups ranges from 1 to 20 (for example, 2, 3, 4, 5, 6, 7, 8, 9, 10, 11, 12, 13, 14, 15, 16, 17, 18, or 19). For the modified redox enzyme of the second type, the number of dehydrogenated amino groups ranges from 1 to 20 (for example, 2, 3, 4, 5, 6, 7, 8, 9, 10, 11, 12, 13, 14, 15, 16, 17, 18, or 19). In other words, the surface of the dehydrogenated redox enzyme can be grafted with 1 to 20 hydrophobic modification chain segments. Multipoint grafting of hydrophobic modification chain segments effectively enhances the ability of the redox enzyme to self-assemble into nanostructures in aqueous solution, thereby improving its dispersion stability and particle size (diameter) control. Moreover, an appropriate number of modification chain segments can provide structural protection without affecting enzymatic activity, thereby improving the enzyme's stability under various pH and temperature conditions. Furthermore, multipoint grafting also helps strengthen the interaction between the enzyme and hydrophobic drugs or carriers, enhancing the efficiency of forming drug complexes and improving functionality in drug delivery, biosensing, and catalytic applications.

It is noteworthy that the modification chain segment can not only serve as a hydrophobic segment to facilitate self-assembly and stabilize the structure, but can also be designed as a fluorescent group due to its fluorescent properties. This enables the resulting enzyme nanoparticles to possess fluorescence characteristics, thereby allowing visualization, tracking, and localization of the enzyme in both in vivo and in vitro environments. Such fluorescent functionality has high application value in the fields of bioimaging, nanoparticle distribution analysis, and site-specific targeting. It assists real-time observation and evaluation of nanoparticle stability, activity retention time, and release dynamics, significantly benefiting quality control and performance optimization of drug delivery systems.

### <Preparation of Modified Redox Enzyme>

The following describes preparation processes of multiple embodiments of the modified redox enzyme of the first type and multiple embodiments of the modified redox enzyme of the second type, to demonstrate the feasibility of the present disclosure. The entire preparation process can be broadly divided into two steps: first, preparing a hydrophobic modification chain segment precursor; then grafting the hydrophobic modification chain segment precursor onto the redox enzyme.

### [Preparation of Modified Redox Enzyme of the First Type]

### Step 1: Preparation of Hydrophobic Modification Chain Segment Precursors (Three Types Listed)

### First Type: 4-piperidinyl-1,8-naphthalimide maleimide

First, 4-bromo-1,8-naphthalic anhydride (20 mmol) and piperidine (26.2 mmol) are dissolved in 2-methoxyethanol (100 ml) and refluxed under nitrogen atmosphere for 24 hours. After cooling to room temperature, the solvent is removed under reduced pressure. The resulting residue is recrystallized with ethanol to obtain orange needle-like crystals of 4-piperidinyl-1,8-naphthalic anhydride, as shown in Formula (A).

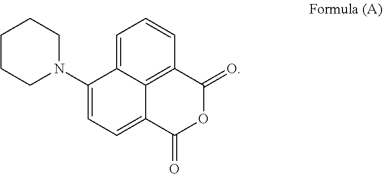 [SMILES: O=C1OC(=O)c2ccc(N3CCCCC3)c3cccc1c23]

Molecule identification of compound of Formula (A): ¹H NMR (300 MHz, CDCl₃, 25° C.): δ=1.70-1.80 (m, 2H; CH₂), 1.85-1.95 (m, 4H; 2CH₂), 3.25-3.35 (m, 4H; 2CH₂), 7.20 (d, J=8.4 Hz, 1H; CH), 7.71 (t, J=7.95 Hz, 1H; CH), 8.44 (dd, J=1, 8.6 Hz, 1H; CH), 8.49 (d, J=8.1 Hz, 1H; CH), 8.57 (dd, J=1, 7.4 Hz, 1H; CH); MS [ESI⁺]: m/z (%): calcd. 282.1, obsvd. 282.31 [M+H]⁺.

Next, dissolve 4-piperidinyl-1,8-naphthalic anhydride (20 mmol) in ethanol (150 ml), then add 6 equivalents of triethylenetetramine. The reaction mixture is refluxed under nitrogen for 1 hour. After cooling to room temperature, the yellow precipitate is filtered to remove the disubstituted byproduct. The filtrate is concentrated under reduced pressure, and the residue is poured into ice water, then extracted with dichloromethane (3 times, 100 ml each). The organic layer is dried over magnesium sulfate, and the solvent is removed under reduced pressure to obtain an orange semi-solid (containing the product 4-piperidinyl-1,8-naphthalimide hexamethyleneamine, as shown in Formula (B)). This product is used directly in the next reaction without purification.

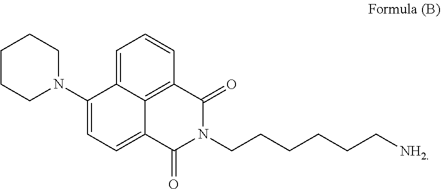 [SMILES: NCCCCCCN1C(=O)c2cccc3c(N4CCCCC4)ccc(c23)C1=O]

Molecule identification of compound of Formula (B): ¹H NMR (500 MHz, CDCl₃, 25° C.): δ=1.35-1.50 (m, 8H; 3CH₂, NH₂), 1.65-1.75 (m, 4H; 2CH₂), 1.80-1.90 (m, 4H; 2CH₂), 2.60-2.70 (m, 2H; CH₂), 3.15-3.25 (m, 4H; 2CH₂), 4.10-4.20 (m, 2H; CH₂), 7.10-7.20 (m, 1H; CH), 7.60-7.70 (m, 1H; CH), 8.35-8.40 (m, 1H; CH), 8.45-8.50 (m, 1H; CH), 8.55-8.60 (m, 1H; CH).

Subsequently, dissolve 4-piperidinyl-1,8-naphthalimide hexamethyleneamine (20 mmol) in dry dichloromethane (50 ml), add 0.35 g of 3-maleimidopropionic acid N-hydroxysuccinimide ester, then add a catalytic amount of pyridine (0.5 ml). The reaction mixture is refluxed under nitrogen for 24 hours. After cooling to room temperature, the solvent is removed under reduced pressure, and the residue is washed with brine and extracted with dichloromethane (3 times, 50 ml each). The organic layer is dried over magnesium sulfate, and the solvent is removed under reduced pressure to obtain a semi-solid material, which is purified by column chromatography to yield a yellow solid product, 4-piperidinyl-1,8-naphthalimide maleimide, which is a hydrophobic modification chain segment precursor according to one embodiment of this disclosure, as shown in Formula (C).

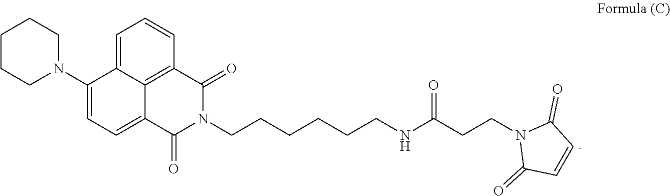 [SMILES: O=C(CCN1C(=O)C=CC1=O)NCCCCCCN1C(=O)c2cccc3c(N4CCCCC4)ccc(c23)C1=O]

Molecule identification of compound of Formula (C): ¹H NMR (600 MHz, Chloroform-d) δ 1.36-1.44 (m, 4H), 1.45-1.52 (m, 2H), 1.67-1.77 (m, 4H), 1.86-1.94 (m, 4H), 2.54 (t, J=7.2 Hz, 2H), 3.17-3.27 (m, 6H), 3.84 (t, J=7.2 Hz, 2H), 4.12-4.18 (m, 2H), 5.89 (s, 1H), 6.69 (s, 2H), 7.19 (d, J=8.1 Hz, 1H), 7.68 (dd, J=8.4, 7.2 Hz, 1H), 8.41 (dd, J=8.4, 1.2 Hz, 1H), 8.48 (d, J=8.1 Hz, 1H), 8.55 (dd, J=7.2, 1.2 Hz, 1H). HRMS [ESI+]: m/z (%): [M+H]⁺ calculated for C₃₀H₃₅N₄O₅ 531.2602; found 531.2603.

### Second Type: N-(2-(3-(2,5-dioxo-2,5-dihydro-1H-pyrrol-1-yl)propanamido)ethyl)-4-(pyren-1-yl)butanamide

First, pyrenebutyric acid (1.7 mmol) and N-tert-butoxycarbonyl ethylenediamine (1.73 mmol) are dissolved in tetrahydrofuran (THF) under an ice bath. Then, N,N-diisopropylethylamine (4.33 mmol) and 1-ethyl-3-(3-dimethylaminopropyl)carbodiimide (2.08 mmol) are sequentially added, and the reaction mixture is stirred overnight. The solvent is removed by rotary evaporation, and the residue is redissolved in chloroform. The solution is extracted with 5% citric acid and saturated brine, and the organic layer is collected and dried over anhydrous magnesium sulfate. The product is purified by column chromatography (eluent starting from methanol:dichloromethane=2:98). The collected product is concentrated by rotary evaporation and dried under vacuum to obtain (2-(4-(pyren-1-yl)butanamido)ethyl)carbamate tert-butyl ester, as shown in Formula (D).

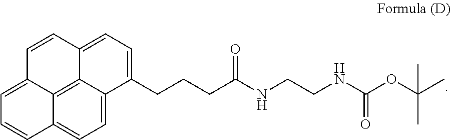 [SMILES: CC(C)(C)OC(=O)NCCNC(=O)CCCc1ccc2ccc3cccc4ccc1c2c34]

Molecule identification of compound of Formula (D): ¹H NMR (600 MHz, DMSO-d6) δ 8.38 (d, J=9.2 Hz, 1H), 8.29-8.25 (m, 2H), 8.22 (dd, J=8.5, 3.4 Hz, 2H), 8.17-8.10 (m, 2H), 8.06 (t, J=7.6 Hz, 1H), 7.94 (d, J=7.8 Hz, 1H), 7.85 (d, J=5.6 Hz, 1H), 6.77 (s, 1H), 3.32-3.29 (m, 2H), 3.09 (t, J=6.2 Hz, 2H), 2.99 (d, J=6.3 Hz, 2H), 2.23 (t, J=7.3 Hz, 2H), 2.01 (dd, J=8.7, 6.6 Hz, 2H), 1.33 (s, 9H). HRMS [ESI+]: m/z (%): calcd. 453.2149, obsvd. 453.2151 [M+Na]+.

Next, (2-(4-(pyren-1-yl)butanamido)ethyl)carbamate tert-butyl ester is dissolved in a mixed solution of 30% trifluoroacetic acid and dichloromethane, stirred at room temperature for 3 hours to remove the tert-butoxycarbonyl protecting group. After the reaction is complete, the solvent is completely removed by nitrogen blowing, then washed with ether to remove impurities. The solid is collected by filtration, and the solid is subjected to rotary evaporation and vacuum drying to completely remove the solvent, obtaining the product 2-(4-(pyren-1-yl)butylamino)ethylamine, as shown in Formula (E).

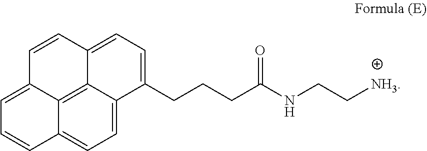

Molecule identification of compound of Formula (E): ¹H NMR (600 MHz, DMSO-d6) δ 8.37 (d, J=9.2 Hz, 1H), 8.24 (t, J=8.3 Hz, 2H), 8.19 (dd, J=8.6, 4.5 Hz, 2H), 8.14-8.07 (m, 3H), 8.03 (t, J=7.6 Hz, 1H), 7.98 (s, 2H), 7.91 (d, J=7.8 Hz, 1H), 3.37-3.29 (m, 4H), 2.91 (t, J=6.4 Hz, 2H), 2.29 (t, J=7.4 Hz, 2H), 2.03 (t, J=7.7 Hz, 2H).

Subsequently, 2-(4-(pyren-1-yl)butylamino)ethylamine (1 equivalent) is dissolved in a mixture of dichloromethane and dimethylformamide, followed by the addition of 0.8 mL of pyridine. Then, 1.5 equivalents of N-hydroxysuccinimide ester of 3-maleimidopropionic acid is added, and the reaction mixture is refluxed overnight. The reaction mixture is poured into diethyl ether to precipitate the crude product, and the solid is collected. The product is then purified by column chromatography and vacuum-dried to obtain N-(2-(3-(2,5-dioxo-2,5-dihydro-1H-pyrrol-1-yl)propanamido)ethyl)-4-(pyren-1-yl) butanamide, which serves as another hydrophobic modification chain segment precursor in an embodiment of the present disclosure, as shown in Formula (F).

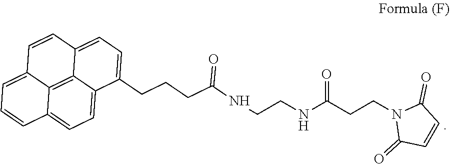 [SMILES: O=C(CCCc1ccc2ccc3cccc4ccc1c2c34)NCCNC(=O)CCN1C(=O)C=CC1=O]

Molecule identification of compound of Formula (F): ¹H NMR (600 MHz, DMSO-d6) δ 8.38 (d, J=9.3 Hz, 1H), 8.27 (td, J=7.5, 1.1 Hz, 2H), 8.23-8.19 (m, 2H), 8.15-8.10 (m, 2H), 8.05 (t, J=7.6 Hz, 1H), 7.99-7.95 (m, 1H), 7.94 (d, J=7.8 Hz, 1H), 7.83 (t, J=5.3 Hz, 1H), 6.96 (s, 2H), 3.59-3.55 (m, 2H), 3.32-3.28 (m, 2H), 3.11-3.04 (m, 4H), 2.32-2.28 (m, 2H), 2.22 (t, J=7.3 Hz, 2H), 2.04-1.98 (m, 2H). HRMS [FD+]: m/z (%): calcd. 481.1996, obsvd. 481.1993 [M].

### Third Type: 3-(2,5-dioxo-2,5-dihydro-1H-pyrrol-1-yl)-N-(2-(2-(naphthalen-2-yl) acetamido)ethyl)propanamide

First, 2-naphthylacetic acid (3.61 mmol), N-tert-butoxycarbonyl ethylenediamine (3.34 mmol), 1-hydroxybenzotriazole (3.613 mmol), and 1-ethyl-3-(3-dimethylaminopropyl)carbodiimide (4.213 mmol) are dissolved in tetrahydrofuran (THF), followed by the addition of N,N-diisopropylethylamine (9.05 mmol). The reaction mixture is stirred for 17 hours. The solvent is removed by rotary evaporation, and the residue is redissolved in ethyl acetate. The organic phase is sequentially washed with 1 M hydrochloric acid, water, saturated sodium bicarbonate, water, and saturated brine. The organic layer is collected, dried over anhydrous magnesium sulfate, and concentrated under reduced pressure and vacuum to obtain the product (2-(2-(naphthalen-2-yl)acetamido)ethyl)carbamate tert-butyl ester, as shown in Formula (G).

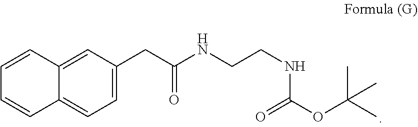 [SMILES: CC(C)(C)OC(=O)NCCNC(=O)Cc1ccc2ccccc2c1]

Molecule identification of compound of Formula (G): ¹H NMR (600 MHz, DMSO-d6) δ 8.10 (t, J=5.7 Hz, 1H), 7.88-7.82 (m, 3H), 7.74 (d, J=1.8 Hz, 1H), 7.51-7.44 (m, 2H), 7.42 (dd, J=8.4, 1.8 Hz, 1H), 6.79 (t, J=5.8 Hz, 1H), 3.57 (s, 2H), 3.10 (q, J=6.4 Hz, 2H), 3.00 (q, J=6.4 Hz, 2H), 1.37 (s, 9H).

Subsequently, (2-(2-(naphthalen-2-yl)acetamido)ethyl)carbamate tert-butyl ester is dissolved in a mixed solution of 30% trifluoroacetic acid and dichloromethane, and stirred at room temperature for 3 hours to remove the tert-butoxycarbonyl protecting group. Upon completion of the reaction, the solvent is completely removed using a nitrogen stream. The residue is rinsed with diethyl ether to remove impurities, and the solid is collected by filtration. The collected solid is further concentrated by rotary evaporation and dried under vacuum to obtain a white solid product, 2-(2-(naphthalen-2-yl)acetamido) ethan-1-amine, as shown in Formula (H).

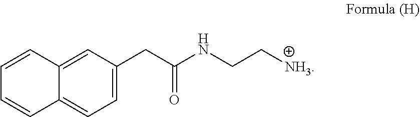

Molecule identification of compound of Formula (H): ¹H NMR (600 MHz, DMSO-d6) δ 8.35 (t, J=5.8 Hz, 1H), 7.93 (s, 3H), 7.86 (ddd, J=16.7, 8.2, 2.0 Hz, 3H), 7.76 (d, J=1.8 Hz, 1H), 7.52-7.42 (m, 3H), 3.62 (s, 2H), 3.31 (q, J=6.3 Hz, 2H), 2.89 (t, J=6.5 Hz, 2H).

Subsequently, 2-(2-(naphthalen-2-yl)acetamido)ethan-1-amine (1.84 mmol) is dissolved in a mixture of dichloromethane and dimethylformamide, followed by the addition of 0.8 mL of pyridine. Then, 3-maleimidopropionic acid N-hydroxysuccinimide ester (2.76 mmol) is added, and the reaction mixture is refluxed overnight. Upon completion, the reaction mixture is poured into diethyl ether to precipitate the crude product. The solid is collected and purified by column chromatography, followed by vacuum drying to obtain the product, 3-(2,5-dioxo-2,5-dihydro-1H-pyrrol-1-yl)-N-(2-(2-(naphthalen-2-yl)acetamido)eth yl)propanamide, which serves as yet another hydrophobic modification chain segment precursor in one embodiment of the present disclosure, as shown in Formula (I).

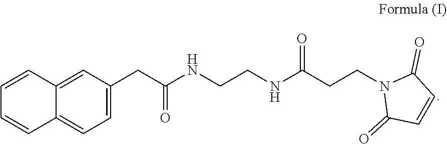 [SMILES: O=C(CCN1C(=O)C=CC1=O)NCCNC(=O)Cc1ccc2ccccc2c1]

Molecule identification of compound of Formula (I): ¹H NMR (600 MHz, DMSO-d6) δ 8.07 (s, 1H), 7.98 (s, 1H), 7.88-7.82 (m, 3H), 7.74 (t, J=1.2 Hz, 1H), 7.51-7.44 (m, 2H), 7.42 (dd, J=8.4, 1.8 Hz, 1H), 7.00 (s, 2H), 3.61-3.56 (m, 4H), 3.07 (td, J=5.9, 5.1, 2.7 Hz, 4H), 2.32-2.28 (m, 2H). HRMS [ESI+]: m/z (%): calcd. 402.1424, obsvd. 402.1426 [M+Na]+.

### Step 2: Preparation of Modified Glucose Oxidase

In this step, the hydrophobic modified chain segment precursor obtained from Step 1 is grafted onto the surface of glucose oxidase (abbreviated as GOx). Specifically, glucose oxidase (0.05 g) is dissolved in 14 mL of phosphate buffer solution and continuously stirred at room temperature in a 100 mL round-bottom flask. Subsequently, the hydrophobic modified chain segment precursor (approximately 10 molar equivalents relative to GOx) is dissolved in 7 mL of dimethylformamide (DMF), and added dropwise (1 mL each time, every 5 minutes) into the stirring GOx solution. The entire reaction proceeds at room temperature under light-protected conditions for 48 hours. After the reaction, the solution is transferred into a dialysis membrane tube with a molecular weight cut-off (MWCO) of 12.4 kDa for dialysis to remove excess hydrophobic modified chain segment precursor and DMF. Fresh deionized water is replaced every 12 hours, and dialysis is conducted for a total of three days. Then, the solvent is removed by lyophilization to obtain GOx grafted with the hydrophobic modified chain segment (i.e., the modified glucose oxidase, represented by Formula (J), wherein GOx′ indicates GOx lacking one SH group), resulting in a yellow solid (approximately 21 mg). The modified glucose oxidase should be stored protected from light at 4° C.

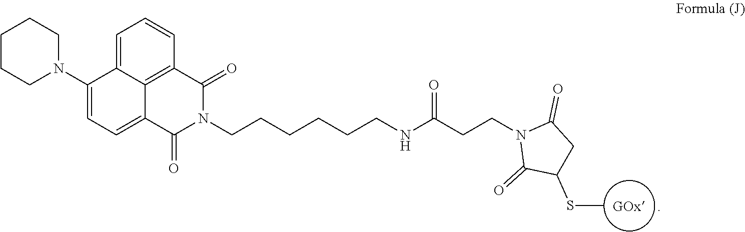 [SMILES: CSC1CC(=O)N(CCC(=O)NCCCCCCN2C(=O)c3cccc4c(N5CCCCC5)ccc(c34)C2=O)C1=O]

### [Preparation of Modified Redox Enzyme of the Second Type]

### Step 1: Preparation of Hydrophobic Modification Chain Segment Precursors (Two Types Listed)

### First Type: 6-(1,3-dioxo-6-(piperidin-1-yl)-1H-benzo[de]isoquinolin-2(3H)-yl)-2-octanamidohexanoic acid

First, 4-bromo-1,8-naphthalic anhydride (5 mmol) and piperidine (10 mmol) are mixed and dissolved in 50 ml chlorobenzene, stirred under reflux for 8 hours. The reaction progress is monitored by thin-layer chromatography (TLC). After cooling to room temperature, extraction is performed with dichloromethane and brine, followed by drying over anhydrous magnesium sulfate. The solvent is then removed by rotary evaporation under reduced pressure, and methanol is added to recrystallize a yellow needle-like solid product, 4-piperidinyl-1,8-naphthalic anhydride, as shown in Formula (K).

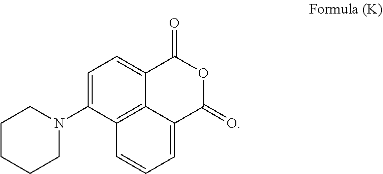 [SMILES: O=C1OC(=O)c2ccc(N3CCCCC3)c3cccc1c23]

Molecule identification of compound of Formula (K): ¹H NMR (300 MHz, [D6] DMSO): δ=1.65-1.80 (m, 2H, CH₂), 1.8-1.95 (m, 4H, CH₂), 3.30 (t, J=5.3 Hz, 4H, CH₂), 4.75 (s, 2H, CH₂), 7.37 (dd, J=7.5, 7.2 Hz, 1H, CH), 7.88 (dd, J=8.0 Hz, 1H, CH), 8.43 (d, J=8.1 Hz, CH), 8.45-8.60 (m, 2H, CH).

Next, 2-chlorotrityl chloride resin (1 g, hereinafter simply referred to as resin) is swollen in anhydrous dichloromethane for 40 minutes. Then, Nα-fluorenylmethyloxycarbonyl-Nε-tert-butoxycarbonyl-L-lysine (Fmoc-lysine (Boc)) (2.00 mmol) and N,N-diisopropylethylamine (6.8 mmol) are mixed and dissolved in an appropriate amount of anhydrous N,N-dimethylformamide (DMF). This solution is added to the apparatus and resin and reacted for 1 hour to allow the amino acid to bind to the resin. The resin is then washed with anhydrous DMF three times (at least 2 minutes each time). Next, a mixture of anhydrous dichloromethane (8 ml), methanol (1.5 ml), and N,N-diisopropylethylamine (0.5 ml) is added to the reaction and stirred for 30 minutes, followed by washing with anhydrous DMF three times (at least 2 minutes each time). Then, a 20% piperidine in DMF solution is used to react for 30 minutes to remove the Nα-fluorenylmethyloxycarbonyl protecting group, followed by washing twice (2 minutes each). Finally, octanoic acid (3.4 mmol), benzotriazol-1-yl-N,N,N′,N′-tetramethyluronium hexafluorophosphate (HBTU) (2.00 mmol), and N,N-diisopropylethylamine (6 mmol) are dissolved in an appropriate amount of anhydrous DMF. This solution is added to the apparatus and resin to couple with the amino acid. After reacting overnight, the white solid product, octanoyllysine (a peptide derivative), is cleaved from the resin using a mixture of 90% trifluoroacetic acid and 10% deionized water, as shown in Formula (L).

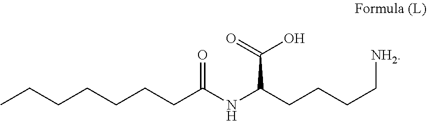

Molecule identification of compound of Formula (L): ¹H NMR (500 MHz, D₂O) δ 4.36 (dd, J=8.8, 4.9 Hz, 1H), 3.00 (t, J=7.5 Hz, 2H), 2.30 (t, J=7.1 Hz, 2H), 1.97-1.88 (m, 1H), 1.82-1.75 (m, 1H), 1.73-1.66 (m, 2H), 1.61 (d, J=5.9 Hz, 2H), 1.47 (dd, J=15.0, 7.2 Hz, 2H), 1.28 (d, J=10.9 Hz, 10H), 0.85 (d, J=6.6 Hz, 3H). HRMS [ESI⁻]: m/z (%): calcd. 272.2100, obsvd. 272.2110 [M−H]⁻.

Next, 4-piperidinyl-1,8-naphthalic anhydride (400 mg) and octanoyllysine (200 mg) are dissolved together with triethylamine (0.05 ml) in 20 ml of ethanol solution, and the mixture is stirred under reflux for 6 hours. The reaction progress is monitored every 2 hours by thin-layer chromatography (TLC). Upon completion of the reaction, the solvent is removed by rotary evaporation. The crude product is purified by column chromatography, wherein 4-piperidinyl-1,8-naphthalic anhydride is first removed using dichloromethane, followed by elution with a dichloromethane:methanol mixture (9:1) to obtain the yellow solid product (6-(1,3-dioxo-6-(piperidin-1-yl)-1H-benzo[de]isoquinolin-2(3H)-yl)-2-octanamidohexanoic acid, which serves as a hydrophobic modification chain segment precursor according to one embodiment of the present disclosure, as shown in Formula (M).

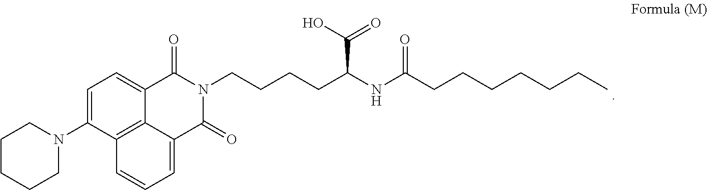 [SMILES: CCCCCCCC(=O)N[C@@H](CCCCN1C(=O)c2cccc3c(N4CCCCC4)ccc(c23)C1=O)C(=O)O]

Molecule identification of compound of Formula (M): ¹H NMR (500 MHz, CDCl₃) δ 8.54 (d, J=6.7 Hz, 1H), 8.47 (d, J=7.3 Hz, 1H), 8.38 (d, J=8.3 Hz, 1H), 7.66 (s, 1H), 7.16 (d, J=7.9 Hz, 1H), 6.63 (s, 1H), 4.45 (s, 1H), 4.18 (d, J=21.9 Hz, 2H), 3.23 (s, 4H), 2.27 (dd, J=15.3, 7.6 Hz, 2H), 2.01 (s, 2H), 1.88 (s, 4H), 1.72 (s, 2H), 1.61 (s, 2H), 1.47 (d, J=7.3 Hz, 2H), 1.32-1.08 (m, 10H), 0.83 (d, J=6.9 Hz, 3H). ¹³C NMR (125 MHz, DMSO-d₆) δ 172.17, 163.54, 163.00, 156.68, 132.23, 130.54, 129.16, 125.79, 125.45, 122.52, 115.02, 114.90, 53.95, 51.71, 45.55, 40.02, 39.85, 39.69, 39.52, 39.35, 39.19, 39.02, 35.02, 31.15, 30.82, 28.46, 28.34, 27.26, 25.70, 25.61, 25.22, 23.83, 23.04, 22.01, 13.91, 8.80. HRMS [ESI⁻]: m/z (%): calcd. 534.2968, obsvd. 534.2998 [M−H⁻].

### Second Type: (6-(1,3-dioxo-6-(piperidin-1-yl)-1H-benzo[de]isoquinolin-2(3H)-yl)-2-palmitamidohexanoic acid)

First, 4-piperidinyl-1,8-naphthalic anhydride (i.e., Formula (K)) is prepared.

Next, the 2-chlorotrityl chloride resin (1 g, hereinafter referred to as “resin”) is swollen in anhydrous dichloromethane for 40 minutes. Then, Nα-fluorenylmethyloxycarbonyl-Nε-tert-butoxycarbonyl-L-lysine (Fmoc-lysine (Boc)) (2.00 mmol) and N,N-diisopropylethylamine (6.8 mmol) are mixed and dissolved in an appropriate amount of anhydrous N,N-dimethylformamide (DMF), and the solution is added to a custom apparatus containing the resin to react for 1 hour to enable coupling of the amino acid to the resin. The resin is washed three times with anhydrous DMF (each wash at least 2 minutes). Subsequently, a mixture of anhydrous dichloromethane (8 ml), methanol (1.5 ml), and N,N-diisopropylethylamine (0.5 ml) is added to the reaction for 30 minutes, followed by three washes with anhydrous DMF (each wash at least 2 minutes). Next, a 20% piperidine/DMF solution is added to remove the Nα-fluorenylmethyloxycarbonyl protecting group by reaction for 30 minutes, and the resin is washed twice (each wash 2 minutes). Finally, palmitic acid (3.4 mmol), benzotriazol-1-yl-N,N,N,N-tetramethyluronium hexafluorophosphate (HBTU) (2.00 mmol), and N,N-diisopropylethylamine (6 mmol) are dissolved in an appropriate amount of anhydrous DMF, and the resulting solution is added to the apparatus to react with the resin to couple the acid to the amino acid. After reacting overnight, the white solid product (peptide derivative) palmitoyllysine is cleaved from the resin using a mixture of 90% trifluoroacetic acid and 10% deionized water, as represented by Formula (N).

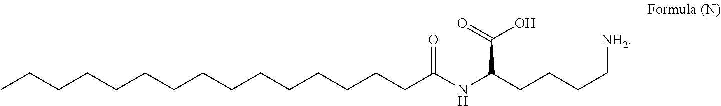

Molecule identification of compound of Formula (N): ¹H NMR (500 MHz, DMSO) δ 12.48 (s, 1H), 7.99 (d, J=7.7 Hz, 1H), 7.70 (s, 2H), 4.15 (s, 1H), 2.76 (s, 2H), 2.10 (t, J=7.0 Hz, 2H), 1.69 (s, 1H), 1.60-1.44 (m, 5H), 1.29 (d, J=51.0 Hz, 26H), 0.85 (d, J=6.4 Hz, 3H). ¹³C NMR (125 MHz, DMSO-d₆) δ 173.71, 172.30, 51.39, 38.61, 35.04, 31.26, 30.47, 29.02, 28.78, 28.67, 28.61, 26.51, 25.22, 22.40, 22.06, 13.92. HRMS [ESI⁻]: m/z (%): calcd. 384.3352, obsvd. 384.3357 [M−H⁻].

Subsequently, 4-piperidinyl-1,8-naphthalic anhydride (400 mg) and palmitoyllysine (200 mg) are dissolved together with triethylamine (0.05 ml) in 20 ml of ethanol and stirred under reflux for 6 hours. The reaction is monitored every 2 hours using thin-layer chromatography. Upon completion, the solvent is removed using a rotary evaporator under reduced pressure. The crude product is purified by column chromatography: 4-piperidinyl-1,8-naphthalic anhydride is first eluted with dichloromethane, followed by elution with a dichloromethane:methanol mixture (9:1) to yield the yellow solid product, 6-(1,3-dioxo-6-(piperidin-1-yl)-1H-benzo[de]isoquinolin-2(3H)-yl)-2-palmitamidohexanoic acid. This compound serves as another hydrophobic modification chain segment precursor according to the present disclosure, as represented by Formula (O).

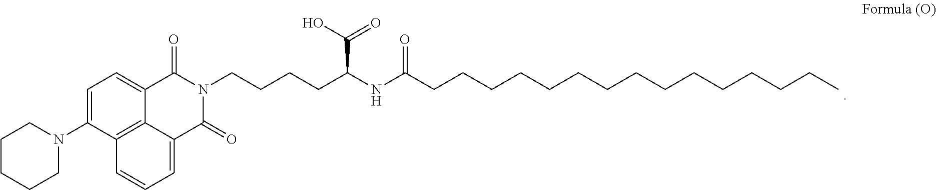 [SMILES: CCCCCCCCCCCCCCCC(=O)N[C@@H](CCCCN1C(=O)c2cccc3c(N4CCCCC4)ccc(c23)C1=O)C(=O)O]

Molecule identification of compound of Formula (O): ¹H NMR (500 MHz, CDCl₃) δ 8.53 (d, J=6.8 Hz, 1H), 8.45 (d, J=8.1 Hz, 1H), 8.36 (d, J=7.9 Hz, 1H), 7.65 (s, 1H), 7.15 (d, J=7.9 Hz, 1H), 6.62 (s, 1H), 4.44 (s, 1H), 4.21-4.10 (m, 2H), 3.22 (s, 4H), 2.31-2.19 (m, 3H), 1.99 (s, 2H), 1.87 (d, J=4.7 Hz, 5H), 1.72 (s, 3H), 1.59 (d, J=7.2 Hz, 3H), 1.47 (d, J=7.2 Hz, 3H), 1.33-1.07 (m, 26H), 0.87 (dd, J=8.4, 5.5 Hz, 3H). ¹³C NMR (125 MHz, DMSO-d₆) δ=172.15, 163.53, 162.99, 156.68, 132.22, 130.54, 129.17, 125.78, 125.46, 122.52, 115.03, 114.89, 53.96, 51.65, 45.56, 40.02, 39.85, 39.69, 39.52, 39.35, 39.19, 39.02, 35.01, 31.24, 30.81, 29.02, 28.97, 28.68, 28.65, 28.47, 27.24, 25.70, 25.22, 23.83, 23.04, 22.04, 13.90, 8.81. HRMS [ESI⁻]: m/z (%): calcd. 646.4220, obsvd. 646.4225 [M−H⁻].

### Step 2: Preparation of Modified Glucose Oxidase

In this step, the hydrophobic modification chain segment precursor obtained in Step 1 is grafted onto the surface of glucose oxidase (abbreviated as GOx). Specifically, a hydrophobic modification chain segment precursor (Formula (M): 30 mg/Formula (O): 36 mg) and sodium bicarbonate (6 mg) are dissolved together in 2 mL of water and stirred at room temperature for 10 minutes. Then, phosphate buffer solution (pH=7.54, 4 mL) is added, followed by the sequential addition of N,N-diisopropylcarbodiimide (12 mg) and hydroxybenzotriazole (8.6 mg), with the mixture stirred at room temperature for 1.5 hours. Glucose oxidase (50 mg) is then added and the reaction continues under stirring at room temperature for 24 hours. Finally, the mixture is dialyzed for two days to remove impurities, and the resulting product is freeze-dried to obtain the solid product of modified glucose oxidase (represented by Formula (P), where GOx′ denotes a GOx molecule lacking one NH₂ group).

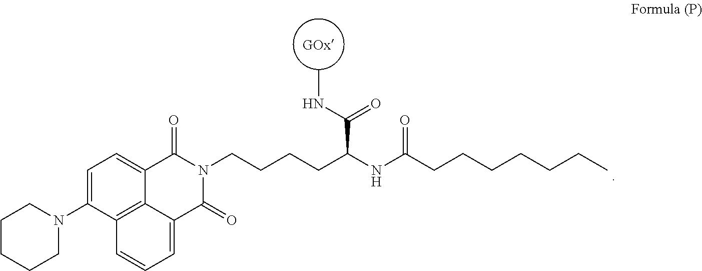 [SMILES: CCCCCCCC(=O)N[C@@H](CCCCN1C(=O)c2cccc3c(N4CCCCC4)ccc(c23)C1=O)C(=O)NC]

### <Nanoparticle Composition>

The modified redox enzyme disclosed herein, due to the grafting of hydrophobic moieties, exhibits amphiphilic molecular characteristics that facilitate self-assembly into multifunctional enzyme nanoparticles, thereby forming the nanoparticle composition of the present disclosure. Specifically, the nanoparticle composition includes a plurality of enzyme nanoparticles, each of which is formed by crosslinking a plurality of the modified redox enzymes with a plurality of aldehyde or aldonic acid molecules. In detail, the aldehyde or aldonic acid molecules undergo Schiff-base reactions with amino groups exposed on the surface of the modified redox enzymes, thereby forming Schiff-base linkages that enable crosslinking among the modified redox enzymes, which in turn aggregate and stabilize into enzyme nanoparticles. The enzyme nanoparticles exhibit enhanced catalytic stability and activity, maintaining functionality under various environmental conditions over extended periods, and significantly improving enzyme utilization efficiency. These features make them suitable for a wide range of biomedical and industrial applications.

In some embodiments, the modified redox enzyme is placed into an aldehyde or aldonic acid solution having a concentration ranging from 0.1 mM to 0.31 mM (e.g., 0.11 mM, 0.13 mM, 0.15 mM, 0.17 mM, 0.19 mM, 0.21 mM, 0.23 mM, 0.25 mM, 0.27 mM, or 0.29 mM) to undergo a crosslinking reaction and form enzyme nanoparticles. During the crosslinking process, the concentration of the aldehyde or aldonic acid molecules in the solution modulates the crosslinking density and particle size of the resulting enzyme nanoparticles, thereby influencing their structural stability and catalytic performance. Specifically, when the concentration of aldehyde or aldonic acid molecules is relatively low, the degree of crosslinking between the modified redox enzymes is limited, resulting in enzyme nanoparticles with larger sizes, looser structures, and relatively lower catalytic activity. As the concentration increases, the crosslinking reaction intensifies, leading to tighter aggregation of the modified redox enzymes, whereby the resulting enzyme nanoparticles become smaller in size and exhibit improved catalytic activity and stability. However, when the concentration of aldehyde or aldonic acid molecules exceeds a certain threshold, excessive crosslinking induces over-aggregation among enzyme nanoparticles, which causes an increase in particle size again and adversely affects their catalytic efficiency and stability.

In some embodiments, the aldehyde or aldonic acid solution includes monocarbonyl aldehydes, dialdehydes, aromatic aldehydes, α,β-unsaturated aldehydes, hydroxy aldehydes, keto aldehydes, aldonic acids, or combinations thereof. In other words, the aldehyde or aldonic acid molecules in the solution may be monocarbonyl aldehydes, dialdehydes, aromatic aldehydes, α,β-unsaturated aldehydes, hydroxy aldehydes, keto aldehydes, aldonic acids, or combinations thereof. In some embodiments, the aldehyde or aldonic acid solution may include aldehyde or aldonic acid molecules of

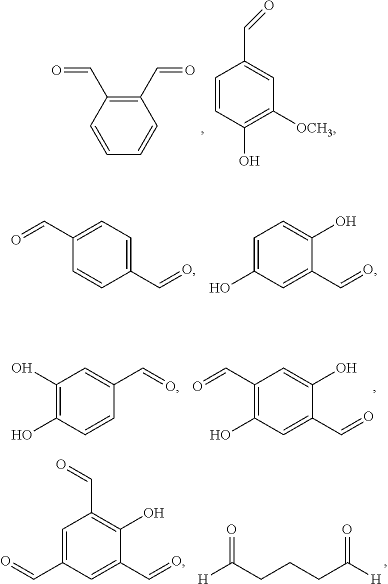

combinations thereof.

In some embodiments, the average diameter of the enzyme nanoparticles may range from 10 nm to 5000 nm (for example, 50 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 1000 nm, 1100 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 4500 nm). The size range of these enzyme nanoparticles provides excellent biocompatibility and tunability, allowing adjustment according to specific application requirements. Smaller enzyme nanoparticles provide a higher specific surface area, thereby enhancing catalytic efficiency and reaction rate; whereas larger enzyme nanoparticles improve stability and durability, suitable for long-term enzymatic reactions. Furthermore, controllability of particle size also helps optimize enzyme stability under different environments, further expanding its applications in drug synthesis, biosensing, environmental technologies, and other fields.

In some embodiments, when glucose oxidase is used as the redox enzyme to prepare the modified redox enzyme (i.e., when the dehydrogenated redox enzyme is formed via dehydrogenation by glucose oxidase), the nanoparticle composition includes a catalytic efficiency (K_{cat}/K_{M}) for glucose greater than 0.5×10⁴ s⁻¹·M⁻¹ and less than 5×10⁴ s⁻¹·M⁻¹, where K_{cat} is the catalytic constant and K_{M} is the Michaelis constant. For example, the catalytic efficiency of the nanoparticle composition for glucose may be 10⁴ s⁻¹·M⁻¹, 1.5×10⁴ s⁻¹·M⁻¹, 2×10⁴ s⁻¹·M⁻¹, 2.5×10⁴ s⁻¹·M⁻¹, 3×10⁴ s⁻¹·M⁻¹, 3.5×10⁴ s⁻¹·M⁻¹, 4×10⁴ s⁻¹·M⁻¹, or 4.5×10⁴ s⁻¹M⁻¹. Specifically, K_{cat} represents the number of substrate molecules catalyzed per active site per unit time and serves as a core indicator to evaluate enzyme catalytic ability; K_{M} represents the enzyme's affinity for the substrate, where a lower value indicates easier complex formation between enzyme and substrate; the ratio K_{cat}/K_{M} is an important indicator for evaluating enzyme performance at low substrate concentrations, with higher values indicating greater efficiency and selectivity. Thus, the enzyme nanoparticles not only maintain the high activity of the native enzyme but also possess improved stability and high substrate affinity after modification, making them particularly suitable for applications in biotransformation, biosensing, and medical diagnostics that require extremely high reaction rates and selectivity.

In some embodiments, when glucose oxidase is used as the redox enzyme to prepare the modified redox enzyme (i.e., when the dehydrogenated redox enzyme is formed via dehydrogenation by glucose oxidase), the nanoparticle composition remains stable under conditions of pH 5 to 9 (e.g., 6, 7, 8) and temperature 15° C. to 60° C. (e.g., 20° C., 25° C., 30° C., 35° C., 40° C., 45° C., 50° C., or 55° C.). Within this pH and temperature range, the catalytic efficiency K_{cat}/K_{M} of the nanoparticle composition for glucose is greater than 0.5×10⁴ s⁻¹·M⁻¹ and less than 5×10⁴ s⁻¹·M⁻¹ (e.g., 10⁴ s⁻¹·M⁻¹, 1.5×10⁴ s⁻¹·M⁻¹, 2×10⁴ s⁻¹·M⁻¹, 2.5×10⁴ s⁻¹·M⁻¹, 3×10⁴ s⁻¹·M⁻¹, 3.5×10⁴ s⁻¹·M⁻¹, 4×10⁴ s⁻¹·M⁻¹, or 4.5×10⁴ s⁻¹·M⁻¹).

### <Preparation of Nanoparticle Composition>

The preparation processes of a plurality of embodiments of the nanoparticle composition are respectively described below to demonstrate the practicability of the present disclosure.

### [Preparation of Nanoparticle Composition Using Modified Redox Enzyme of the First Type]

The modified glucose oxidase (2 mg) of the first type is dissolved in deionized water (200 μl). Ethanol is then gradually added (5 μl each time) until the solution becomes turbid. Glutaraldehyde is added, and the mixture is stirred at room temperature for 1.5 hours. After the reaction, the formed nanoparticles are collected by centrifugation (12,000 g, 15 minutes). The collected precipitate is resuspended in a mixture of ethanol and deionized water, followed by centrifugation under the same conditions. Subsequently, the nanoparticles are washed twice by centrifugation under the same conditions using ultrapure water. Finally, the precipitate is resuspended in deionized water and stored at 4° C. until use.

### [Preparation of Nanoparticle Composition Using the Modified Redox Enzyme of the Second Type]

10 milligrams of the modified glucose oxidase of the second type is gradually added to 2 mL of isopropanol (200 μL at a time, with thorough stirring after each addition). Once the solution becomes turbid, 20 μL of 8% glutaraldehyde is added to crosslink the modified redox enzyme, and the mixture is stirred at room temperature for 12 hours. The resulting product is then separated by centrifugation (12,000 g, 15 minutes). The supernatant is removed, and the same solvent (isopropanol) is added, followed by another round of centrifugation; this step is repeated twice. The resulting precipitate is then resuspended in isopropanol using an ultrasonic bath and stored at 4° C. until use.

### <Nanomedicine>

The disclosed nanoparticle composition may be applied in the development of nanomedicines. Specifically, the nanomedicine may include the aforementioned nanoparticle composition, which is capable of actively exhibiting enzymatic activity in vivo to catalyze specific substrates. In some embodiments, when glucose oxidase is used as the redox enzyme to prepare the modified redox enzyme, the resulting enzyme nanoparticles may catalyze the decomposition of glucose within the tumor microenvironment, thereby generating gluconic acid and hydrogen peroxide (H₂O₂), effectively cutting off the glucose supply to tumor cells and thereby inhibiting their growth. Since glucose is a key nutrient source for tumor cell metabolism, the metabolic deprivation effect achieved by glucose consumption can induce an anti-cancer effect via starvation therapy, selectively destroying tumor cells and suppressing their proliferation. Notably, the H₂O₂ generated from glucose decomposition can further produce highly reactive hydroxyl radicals (—OH), which induce oxidative damage to tumor cells. Based on the foregoing, the disclosed nanomedicine can be combined with various therapeutic approaches, such as phototherapy, gas therapy, chemotherapy, catalytic therapy, and immunotherapy, to achieve multimodal synergistic therapeutic effects.

### <Nanomedicine Complex>

The present disclosure also provides a nanomedicine complex, which includes the aforementioned nanoparticle composition and a drug molecule, wherein the drug molecule is bonded to the modified redox enzyme in the nanoparticle composition in a releasable form. Through the active catalysis of substrate reactions by the nanoparticle composition at a target site and the alteration of the tumor microenvironment during this process, the drug molecule can be released, thereby achieving drug delivery while simultaneously inhibiting the nutrient supply to tumor cells. This enables a dual therapeutic effect combining drug therapy and enzyme therapy. In some embodiments, the drug molecule may be an anticancer drug and may be conjugated to the nanoparticle composition via appropriate means such as covalent bonding, electrostatic adsorption, hydrophobic interaction, or π-π stacking. For example, hydrophilic drugs may be encapsulated in the aqueous core of the enzyme nanoparticles, whereas hydrophobic drugs may be embedded in the hydrophobic regions of the enzyme nanoparticles. This design enables stable encapsulation and controlled release of the drug molecules, enhances their circulation time in the body and accumulation in tumor tissues, thereby improving therapeutic efficacy and reducing side effects.

### <Preparation of the Nanomedicine Complex>

In some embodiments, during the preparation process of the nanomedicine complex, drug molecules may first be added to a solution of the modified redox enzyme prior to self-assembly. Aldehyde or aldonic acid molecules are then added to promote the self-assembly behavior of the modified redox enzyme. Through this co-self-assembly approach, in which the modified redox enzyme and the drug molecules self-assemble together, the drug molecules can be stably encapsulated within the enzyme nanoparticles during the enzyme self-assembly process, thereby forming a stable nanomedicine complex.

The following describes the preparation processes of a plurality of embodiments of the nanomedicine complex, in order to demonstrate the feasibility of the present disclosure.

[Nanomedicine Complex Loaded with Doxorubicin (DOX)]

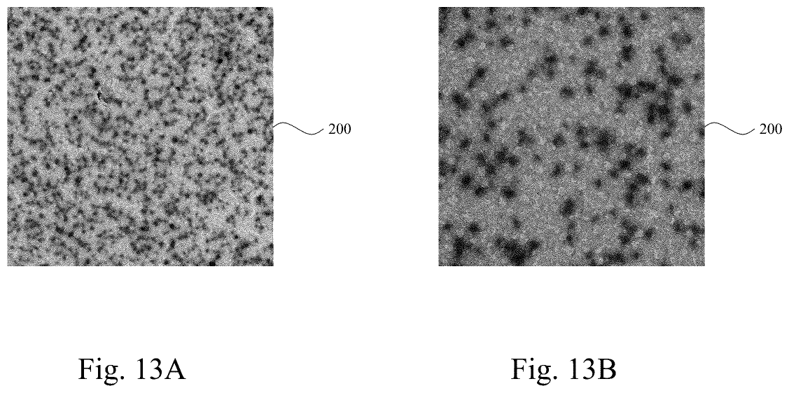

The aforementioned modified glucose oxidase (2 mg) of the second type is dissolved in deionized water (200 μL). Subsequently, 0.1 mg of doxorubicin hydrochloride (dissolved in 20 μL of deionized water) is added with continuous stirring. Ethanol (20 μL at a time) is then gradually added to the modified glucose oxidase solution until the solution becomes turbid. At this point, the ethanol-to-water ratio is approximately 3:2. Glutaraldehyde is then added, and the mixture is stirred at room temperature for 8 hours. The DOX-loaded enzyme nanoparticles are collected by centrifugation (12,000 g, 15 minutes). The resulting precipitate is resuspended in a mixture of ethanol and deionized water and centrifuged again under the same conditions. The particles are then washed twice with ultrapure water under the same conditions. Finally, the DOX-loaded nanomedicine complex is collected and stored at 4° C. Referring to FIGS. 13A-13B, in which FIG. 13A shows a transmission electron microscopy (TEM) image (magnification: 15,000×) of the DOX-loaded nanomedicine complex 200, and FIG. 13B shows a partially enlarged schematic view of FIG. 13A. The TEM image shows that the nanomedicine complex 200 appears as complete and well-defined particulate (spherical) structures, with a measured particle size of 30.46±6.71 nm, confirming that the drug molecules are stably encapsulated into the enzyme nanoparticles during the enzyme self-assembly process, thereby forming a stable nanomedicine complex.

[Nanomedicine Complex Loaded with Paclitaxel (PTX)]

The aforementioned modified glucose oxidase (2 mg) of the second type is dissolved in deionized water (200 μL). Then, 0.1 mg of paclitaxel (dissolved in 20 μL of ethanol) is added with continuous stirring. Ethanol (20 μL at a time) is then gradually added to the modified glucose oxidase solution until the solution becomes turbid. At this point, the ethanol-to-water ratio is approximately 1:2. Glutaraldehyde is subsequently added, and the mixture is stirred at room temperature for 8 hours. The PTX-loaded enzyme nanoparticles are then collected by centrifugation (12,000 g, 15 minutes). The resulting precipitate is resuspended in a mixture of ethanol and deionized water and centrifuged again under the same conditions. The particles are then washed twice with ultrapure water under the same conditions. Finally, the PTX-loaded nanomedicine complex is collected and stored at 4° C.

### <Electrical Signal Sensor>

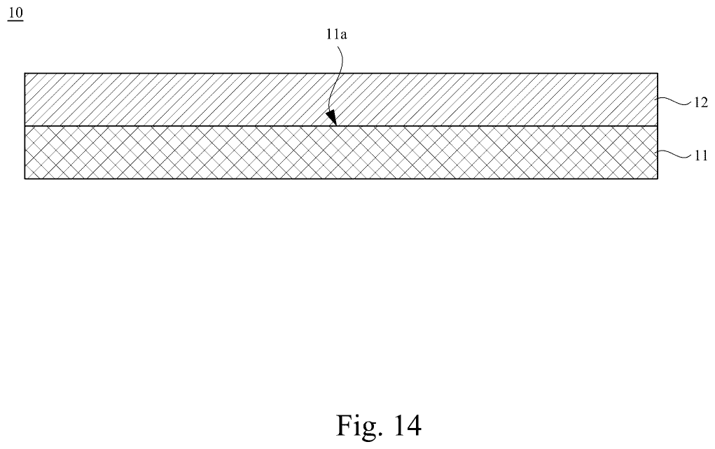

The modified redox enzyme and the nanoparticle composition disclosed herein are also applicable to the development of an electrical signal sensor. In detail, please refer to FIG. 14, which is a side schematic view schematic diagram of an electrical signal sensor 10 according to some embodiments of the present disclosure. The electrical signal sensor 10 includes an electrode layer 11 and a sensing layer 12 disposed on a surface 11a of the electrode layer 11, wherein the sensing layer 12 includes the aforementioned modified redox enzyme or nanoparticle composition. Such a sensing layer generates an electrochemical signal through a specific catalytic reaction between the modified redox enzyme and a target substrate (e.g., glucose), for example, by generating hydrogen peroxide, which subsequently induces a detectable current change at the electrode. Based on this, the sensor effectively converts changes in substrate concentration into corresponding electrical signals, thereby achieving highly sensitive and highly selective electrochemical detection. In some embodiments, the sensor is applicable to blood glucose monitoring.

Since the nanoparticle composition is formed via a self-assembly mechanism, its high specific surface area and uniform dispersion enable the sensing layer to provide more reactive active sites, thereby effectively increasing the contact probability and reaction efficiency between the enzyme and the substrate. This structural advantage significantly enhances the intensity of the catalytically generated electrical signal and lowers the detection limit of the overall sensing system, allowing detection of target molecules at extremely low concentrations. Additionally, the self-assembled nanoparticles help expand the sensor's working concentration range, covering detection needs from trace amounts to high concentrations of the target, while improving overall sensitivity and reproducibility, and providing high recognition capability for subtle concentration changes. Due to the excellent stability and enzymatic activity of the disclosed modified redox enzyme, and its further enhanced stability and resistance to degradation in the sensing environment after self-assembly into nanoparticle carriers, the lifespan of the sensing layer is extended and background noise is reduced. Overall, this sensing design not only offers highly sensitive and stable electrochemical sensing performance but is also suitable for portable or wearable biosensing devices, possessing potential for long-term, continuous monitoring.

The efficacy of the present disclosure is validated through multiple comparative examples and multiple embodiments described below. It should be understood that the present disclosure is not to be construed as limited by the following embodiments.

### [Experiment 1: Particle Size Analysis of Enzyme Nanoparticles]

In this experiment, a scanning electron microscope (SEM) is used to measure the particle size of enzyme nanoparticles within the nanoparticle composition. Specifically, the modified glucose oxidase is prepared using the method described above in [Preparation of Modified Redox Enzyme](Note: using the hydrophobic modification chain segment precursor of the first type), and the nanoparticle composition is prepared from the modified glucose oxidase using the method described above in [Preparation of Nanoparticle Composition Using Modified Redox Enzyme of the first type]. The correlation between glutaraldehyde concentration and the particle size of enzyme nanoparticles in the nanoparticle composition is then analyzed.

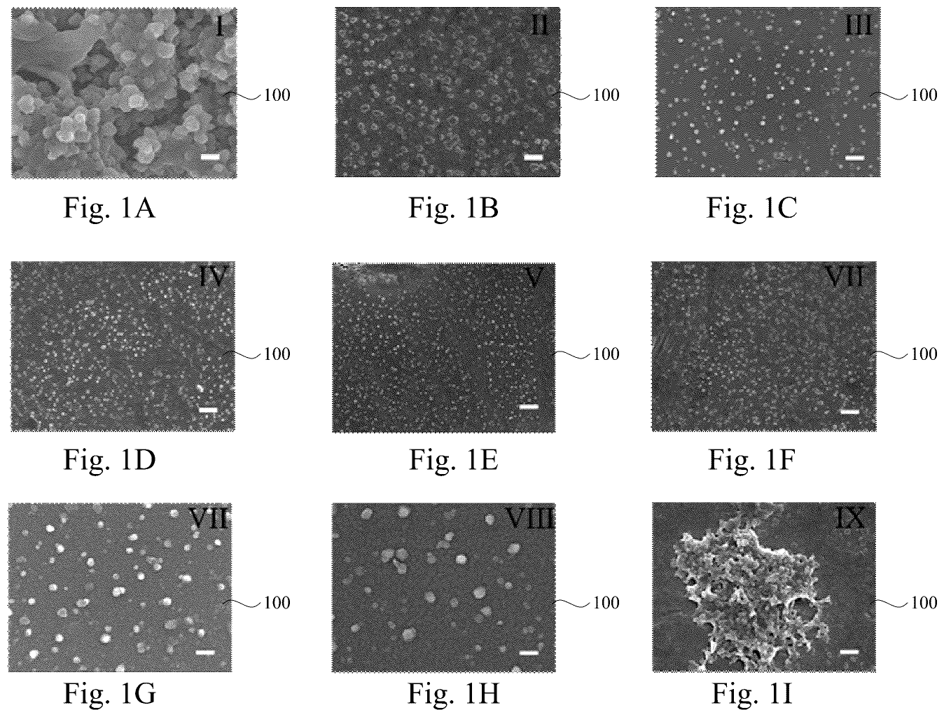

Please refer to FIGS. 1A to 1I for the experimental results, which show scanning electron microscopy (SEM) images of the modified glucose oxidase in glutaraldehyde solutions of different concentrations. Specifically, the glutaraldehyde concentrations in FIGS. 1A to 1I are 0 mM (label I), 0.10 mM (label II), 0.1 mM (label III), 0.2 mM (label IV), 0.21 mM (label V), 0.22 mM (label VI), 0.23 mM (label VII), 0.31 mM (label VIII), and 0.35 mM (label IX), respectively. The scale bar (white line at the lower right corner) in each figure represents 1.0 micrometer. The SEM images show that initially, as the glutaraldehyde concentration increases, the particle size and aggregation degree of the enzyme nanoparticles 100 (white particles in FIGS. 1A to 1I) self-assembled from the modified glucose oxidase gradually decrease; however, when the concentration exceeds 0.21 mM, the particle size and aggregation degree increase. These results indicate that the glutaraldehyde concentration significantly affects the self-assembly behavior of the modified glucose oxidase, and an optimal concentration range exists (e.g., 0.2 mM to 0.23 mM) in which enzyme nanoparticles 100 with smaller particle size and uniform dispersion can be formed.

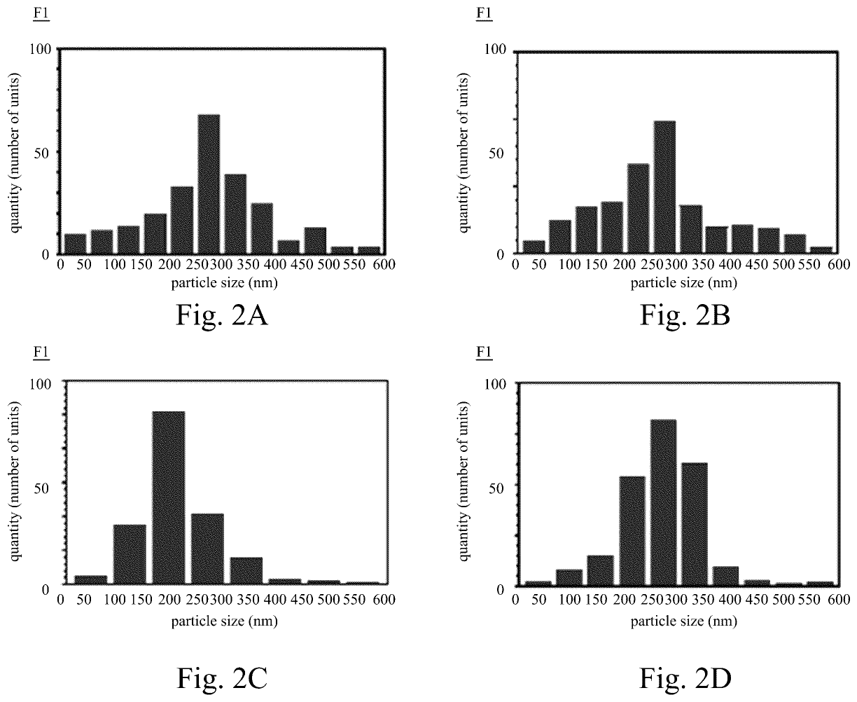

To further verify, dynamic light scattering (DLS) is used to analyze the particle size of enzyme nanoparticles 100 shown in FIGS. 1A to 1I. The experimental results are presented in Table 1, and some results are further shown in FIGS. 2A to 2D, where FIGS. 2A to 2D correspond to the particle size distribution graphs of enzyme nanoparticles 100 in FIGS. 1C to 1F, respectively. The results indicate that the average particle size of enzyme nanoparticles 100 generally decreases as the glutaraldehyde concentration increases, reaching a minimum at a glutaraldehyde concentration of 0.21 mM, which is consistent with the observations from the SEM images.

TABLE 1

| Glutaraldehyde | Average particle size of enzyme |
|---|---|
| concentration (mM) | nanoparticles (nm) |
| 0 | 463.28 ± 23.47 |
| 0.19 | 324.97 ± 21.05 |
| 0.10 | 389.36 ± 15.61 |
| 0.20 | 267.54 ± 27.30 |
| 0.21 | 183.19 ± 40.45 |
| 0.22 | 296.31 ± 45.95 |
| 0.23 | 326.33 ± 20.71 |
| 0.31 | 583.54 ± 35.24 |
| 0.35 | 860.25 ± 73.93 |

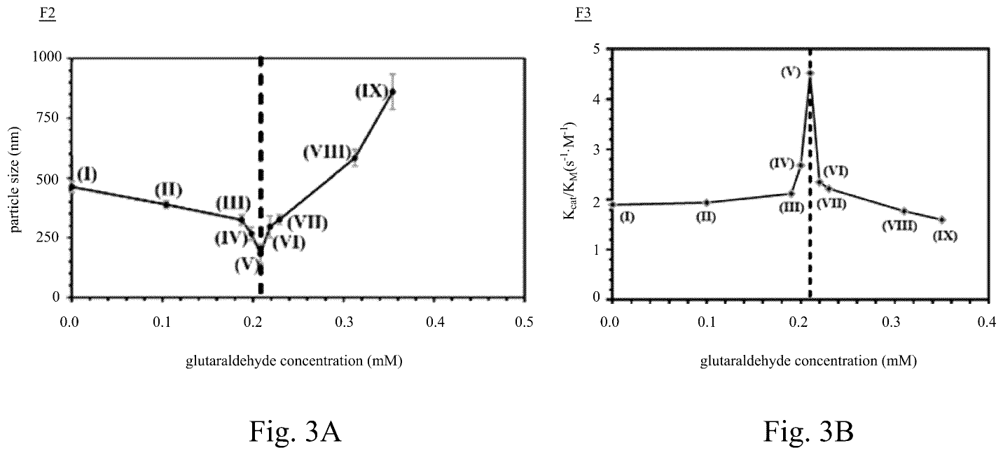

The results in Table 2 are then plotted in FIG. 3A, which is the average particle size curve (F2) of the enzyme nanoparticles 100 in FIG. 1A to FIG. 1H under different glutaraldehyde concentrations (since obvious precipitation occurs in FIG. 1I, concentrations exceeding 0.35 mM are not further studied). The labels (I) to (IX) in FIG. 3A correspond to the labels I to IX in FIG. 1A to FIG. 1H. The results show that the glutaraldehyde concentration and the size of the enzyme nanoparticles exhibit a nonlinear V-shaped relationship. Specifically, at a concentration of 0.21 mM, the average particle size reaches the minimum, decreasing from approximately 475 nm to about 150 nm. It can be seen that at lower concentrations, due to limited crosslinking, the enzyme nanoparticles remain relatively large; as the glutaraldehyde concentration increases, the crosslinking reaction intensifies, and the particle structure becomes more compact, resulting in size reduction; when the concentration exceeds 0.21 mM, excessive crosslinking causes particle aggregation, ultimately leading to an increase in particle size. In summary, the SEM and DLS analyses indicate that a high concentration of glutaraldehyde promotes the formation of enzyme nanoparticles, but an excessive amount of crosslinking agent induces aggregation, making the particles larger, explaining the key role of the interaction between enzyme nanoparticles and glutaraldehyde.

Furthermore, the present disclosure further conducts steady-state kinetic analysis of the nanoparticle composition under different glutaraldehyde concentrations using the Michaelis-Menten and Lineweaver-Burk models. The results are shown in FIG. 3B, which is a reaction rate curve (F3) of the nanoparticle composition at various glutaraldehyde concentrations. The labels (I) to (IX) in FIG. 3B correspond to the labels I to IX in FIG. 1A to FIG. 1H. The experimental results indicate that the catalytic efficiency of the nanoparticle composition exhibits a nonlinear inverted V-shaped relationship with glutaraldehyde concentration. When the glutaraldehyde concentration is 0.21 mM, the particle size of the enzyme nanoparticles is minimized, maximizing the exposure of the enzymatic active sites, thereby resulting in the highest reaction rate. In contrast, at lower concentrations, due to incomplete crosslinking, the larger and looser structure of the enzyme nanoparticles limits enzymatic stability and activity, leading to a lower reaction rate. As the glutaraldehyde concentration continues to increase, excessive crosslinking causes aggregation of the enzyme nanoparticles, hindering access to the active sites and resulting in a decrease in reaction rate. These results demonstrate that reducing the particle size facilitates enhancement of catalytic efficiency, but excessive condensation and aggregation reduce catalytic performance, highlighting the critical importance of controlling the degree of crosslinking in the design of enzyme nanoparticles.

### <Experiment 2: Electrochemical Analysis of Enzyme Nanoparticles>

In this experiment, the modified glucose oxidase is prepared using the aforementioned method for [Preparation of Modified Redox Enzyme of the First Type](note: using the hydrophobic modification chain segment precursor of the first type), and the resulting modified glucose oxidase is further processed into a nanoparticle composition using the method for [Preparation of Nanoparticle Composition Using Modified Redox Enzyme of the first type] described earlier. Amperometric analysis is then performed at a fixed voltage of 0.65 V to measure the current response of the nanoparticle composition under different glutaraldehyde concentrations and varying glucose concentrations (2 mM, 10 mM, and 24 mM).

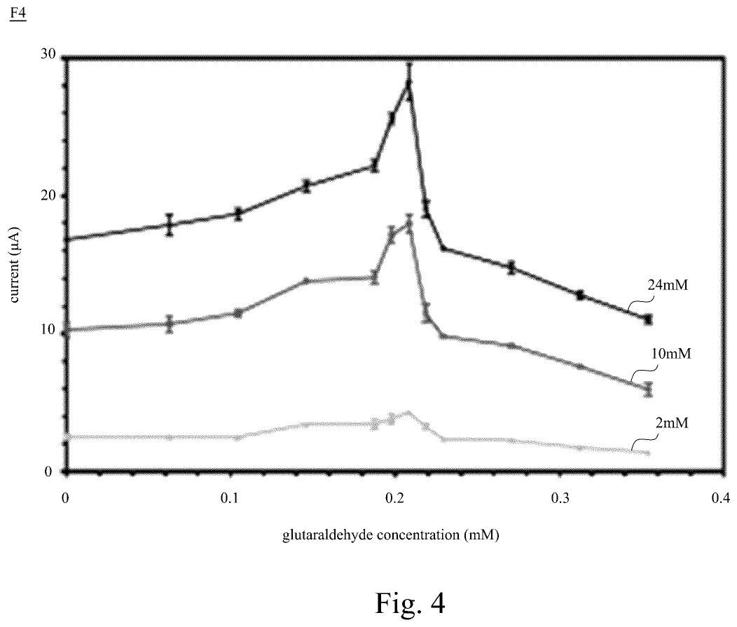

The experimental results are shown in FIG. 4, which presents the current response curve (F4) of the nanoparticle composition under different glutaraldehyde concentrations and varying glucose concentrations. The corresponding experimental data are summarized in Table 2. The results indicate that when the injected glucose concentrations are 2 mM, 10 mM, and 24 mM, the current intensity increases with the rise in glutaraldehyde concentration and reaches a peak at a glutaraldehyde concentration of 0.21 mM. At this optimal concentration (0.21 mM), the corresponding current intensities are 4.3 μA, 18.0 μA, and 28.3 μA, respectively. When the glutaraldehyde concentration exceeds 0.21 mM, the current intensity drops sharply with further increases in the crosslinking agent concentration. This trend exhibits an inverse relationship with the particle size variation observed in the DLS analysis, suggesting that smaller nanoparticles correspond to higher current intensities. This phenomenon is attributed to the increased surface area of the nanoparticles, enabling more efficient enzyme immobilization and enhanced interaction with glucose, thereby improving the sensitivity and signal output of the sensor. These findings further demonstrate the advantages of the nanoparticle composition in electrochemical glucose detection.

TABLE 2

| | Average particle size of enzyme nanoparticles (nm) | | | | | |
|---|---|---|---|---|---|---|
| | 24 | | 10 | | 2 | |
| Glutaraldehyde | Average | | Average | | Average | |
| concentration | Current | Standard | Current | Standard | Current | Standard |
| (mM) | (μA) | Deviation | (μA) | Deviation | (μA) | Deviation |
| 0 | 16.82 | 0.05 | 10.31 | 0.47 | 2.53 | 0.19 |
| 0.06 | 17.89 | 0.75 | 10.72 | 0.59 | 2.54 | 0.13 |
| 0.10 | 18.69 | 0.40 | 11.50 | 0.22 | 2.52 | 0.08 |
| 0.15 | 20.71 | 0.41 | 13.83 | 0.18 | 3.45 | 0.11 |
| 0.19 | 22.18 | 0.44 | 14.11 | 0.45 | 3.45 | 0.33 |
| 0.20 | 25.59 | 0.38 | 17.18 | 0.60 | 3.85 | 0.32 |
| 0.21 | 28.26 | 1.35 | 17.96 | 0.64 | 4.32 | 0.14 |
| 0.22 | 19.05 | 0.58 | 11.48 | 0.65 | 3.27 | 0.23 |
| 0.23 | 16.18 | 0.12 | 9.83 | 0.08 | 2.40 | 0.04 |
| 0.27 | 14.80 | 0.41 | 9.15 | 0.12 | 2.29 | 0.10 |
| 0.31 | 12.81 | 0.32 | 7.63 | 0.04 | 1.77 | 0.18 |
| 0.35 | 11.05 | 0.28 | 5.98 | 0.44 | 1.43 | 0.13 |

### <Experiment 3: Kinetic Analysis of Enzyme Nanoparticles>

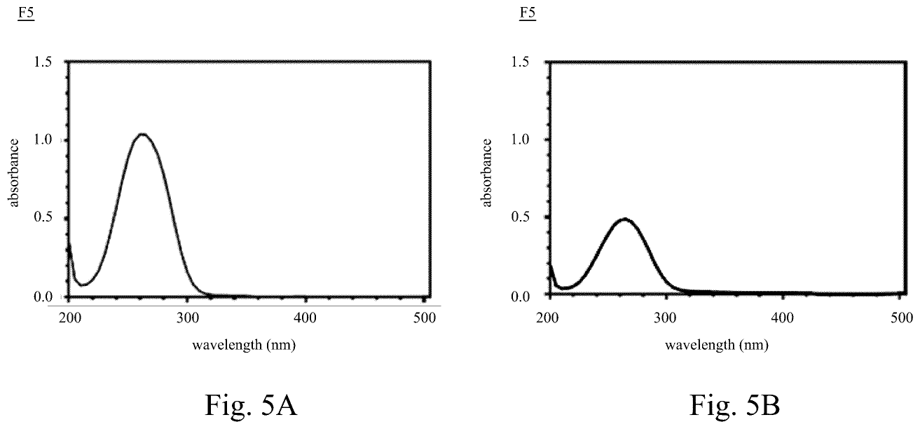

In this experiment, the modified glucose oxidase is prepared using the aforementioned method for [Preparation of Modified Redox Enzyme of the First Type](note: using the hydrophobic modification chain segment precursor of the first type), and the resulting modified glucose oxidase is further processed into a nanoparticle composition using the method for [Preparation of Nanoparticle Composition Using Modified Redox Enzyme of the First Type] described earlier. The UV-Vis absorption spectra of the modified glucose oxidase and the nanoparticle composition are measured under the same enzyme concentration (2 μg/mL) during the catalysis of 10 mM glucose. The experimental results are shown in FIG. 5A and FIG. 5B, which respectively depict the UV-Vis absorption spectra of the nanoparticle composition and the modified glucose oxidase (F5). The absorption peak appears at 250 nm, corresponding to the UV-Vis absorption peak of H₂O₂ as reported in the literature. Furthermore, the nanoparticle composition produces a higher concentration of H₂O₂ upon catalyzing glucose, indicating superior catalytic performance compared to the non-nanostructured modified redox enzyme.

Subsequently, the catalytic efficiency of the nanoparticle composition is investigated using the 2,2′-azino-bis(3-ethylbenzothiazoline-6-sulfonic acid) (ABTS) assay. Specifically, in the cascade reaction involving horseradish peroxidase (HRP) and ABTS, both the modified glucose oxidase and the nanoparticle composition catalyze the oxidation of glucose to glucono-5-lactone and H₂O₂ in the presence of oxygen. The generated H₂O₂ then reacts with HRP to oxidize the colorless ABTS into the green ABTS radical cation (ABTS⁺·), enabling colorimetric analysis of enzyme activity. The catalytic efficiency of the nanoparticle composition is confirmed to be significantly higher, as indicated by the more intense color development of the glucose solution under identical concentrations of glucose, HRP, and ABTS, and the same reaction time.

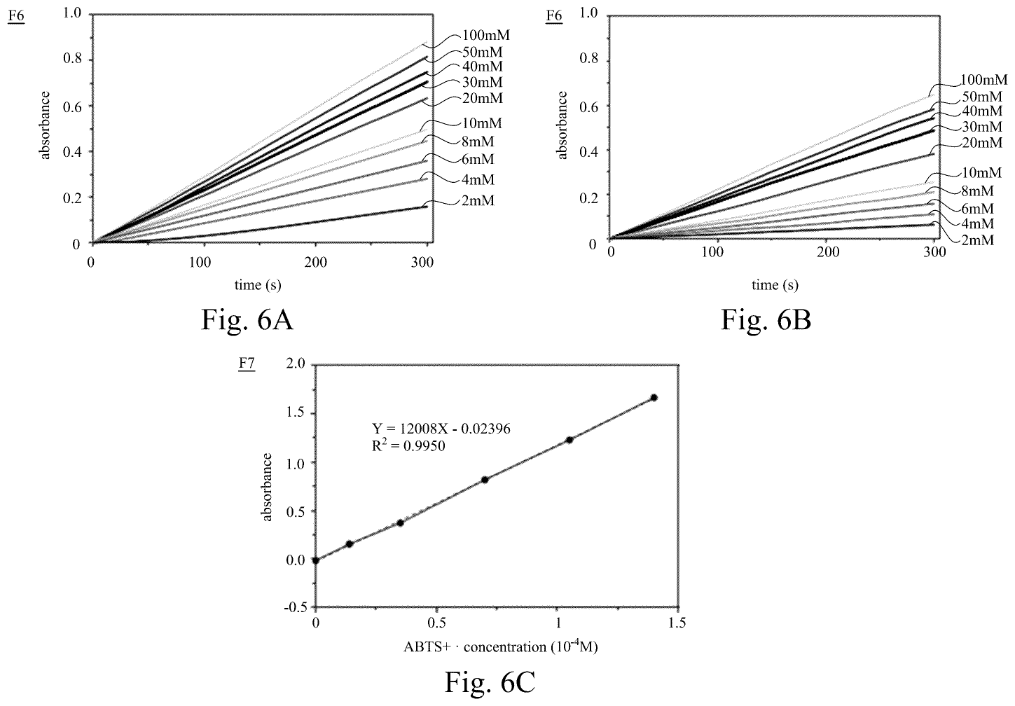

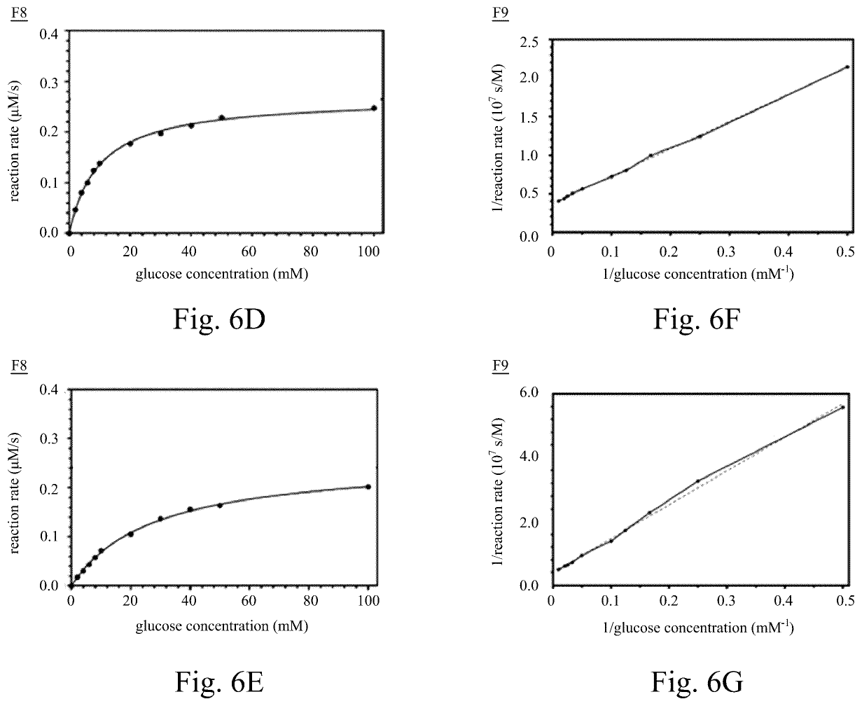

For further analysis, enzyme kinetics are conducted using the Michaelis-Menten curve and the Lineweaver-Burk double reciprocal plot. Specifically, the time-dependent absorbance of ABTS⁺· at 740 nm under various glucose concentrations is measured (FIGS. 6A and 6B), and the absorbance data are converted into ABTS⁺· concentrations (FIG. 6C). The Michaelis-Menten curves of the nanoparticle composition and the modified glucose oxidase are then plotted separately (FIGS. 6D and 6E), followed by conversion into Lineweaver-Burk double reciprocal plots for each (FIGS. 6F and 6G). In detail, FIG. 6A and FIG. 6B respectively show the absorbance-time plots of the nanoparticle composition and the modified glucose oxidase at different glucose concentrations (F6); FIG. 6C shows the absorbance-concentration calibration curve of ABTS⁺· (F7); FIGS. 6D and 6E show the Michaelis-Menten plots of the nanoparticle composition and the modified glucose oxidase, respectively (F8); and FIGS. 6F and 6G show the corresponding Lineweaver-Burk double reciprocal plots (F9). The kinetic parameters (V_{Max}, K_{M}, K_{cat}, and K_{cat}/K_{M}) obtained from the above analyses for both the nanoparticle composition and the modified glucose oxidase are summarized in Table 3. V_{Max} represents the maximum reaction rate under saturated glucose concentration, while K_{M} refers to the glucose concentration at which the reaction rate reaches ½ of V_{Max}. The meanings of K_{cat} and K_{cat}/K_{M} are described previously and will not be reiterated here.

TABLE 3

| | V_{Max} | K_{M} | K_{cat} | K_{cat}/K_{M} |
|---|---|---|---|---|
| | (μM^{s - 1}) | (mM) | (s⁻¹) | (M⁻¹s⁻¹) |
| modified glucose | 0.2411 | 25.49 | 388.81 | 1.49 × 10⁴ |
| oxidase | | | | |
| nanoparticle | 0.2646 | 9.33 | 421.87 | 4.52 × 10⁴ |
| composition | | | | |

The experimental results show that the catalytic efficiency (K_{cat}/K_{M}) of the nanoparticle composition is increased by more than 300% compared to that of the modified glucose oxidase, highlighting its enhanced catalytic capability. Notably, the catalytic efficiency of the disclosed nanoparticle composition is competitive with that of glucose oxidase reported in the literature and is approximately 66 times higher than that of glucose oxidase nanoparticles (676 M⁻¹·s⁻¹).

### <Experiment 4: Analysis of the Effect of pH on the Stability of Enzyme Nanoparticles>

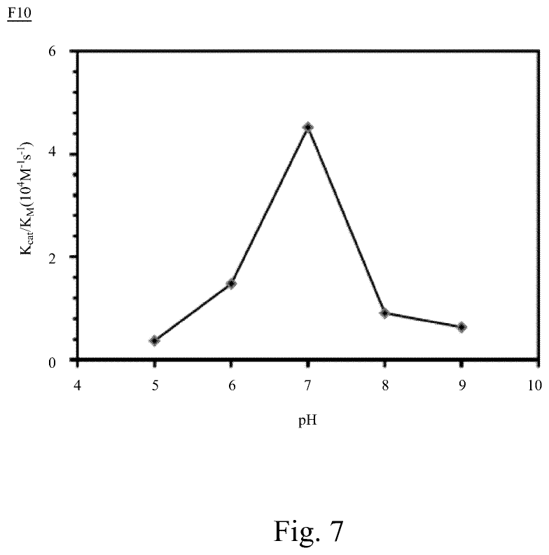

In this experiment, the modified glucose oxidase is prepared using the aforementioned method for [Preparation of Modified Redox Enzyme of the First Type](note: using the hydrophobic modification chain segment precursor of the first type), and is subsequently processed into a nanoparticle composition using the method for [Preparation of Nanoparticle Composition Using Modified Redox Enzyme of the First Type] described earlier. Deionized water solutions with varying pH values are then prepared, into which horseradish peroxidase (HRP, 0.4 μg/mL) and ABTS (2.5 mM) are added, followed by addition of the nanoparticle composition (0.1 μg/mL). 1 mL of the resulting mixture is placed in a UV-Vis spectrophotometer (model V670, JASCO), and the detection wavelength is set to 740 nm, corresponding to the characteristic absorption wavelength of ABTS⁺·. Subsequently, glucose is added, and the time-dependent absorbance is measured. Using the standard calibration curve of ABTS⁺· and the slope of the reaction rate, the time-dependent concentration of ABTS⁺· is calculated. Enzyme kinetic parameters are then determined based on the Michaelis-Menten model, with further analysis performed using the Lineweaver-Burk model to evaluate the steady-state enzymatic behavior of the nanoparticle composition under different pH conditions. The experimental results are shown in FIG. 7, which presents the reaction rate curves (F10) of the nanoparticle composition under different pH values. As shown in FIG. 7, the catalytic efficiency of the nanoparticle composition exhibits a bell-shaped trend across different pH levels, reaching a maximum at pH 7, with a K_{cat}/K_{M} value of approximately 4.8×10⁴ s⁻¹·M⁻¹, indicating optimal enzymatic activity under neutral conditions. Although the catalytic efficiency slightly decreases under acidic (pH 5, K_{cat}/K_{M}=0.5×10⁴ s⁻¹·M⁻¹) and alkaline (pH 9, K_{cat}/K_{M}=1.0×10⁴ s⁻¹·M⁻¹) conditions, the nanoparticle composition overall maintains stable enzymatic activity in the pH range of 5 to 9, demonstrating good pH tolerance and broad applicability.

### <Experiment 5: Quantification of Hydrophobic Modification Chain Segments in the Modified Redox Enzyme>

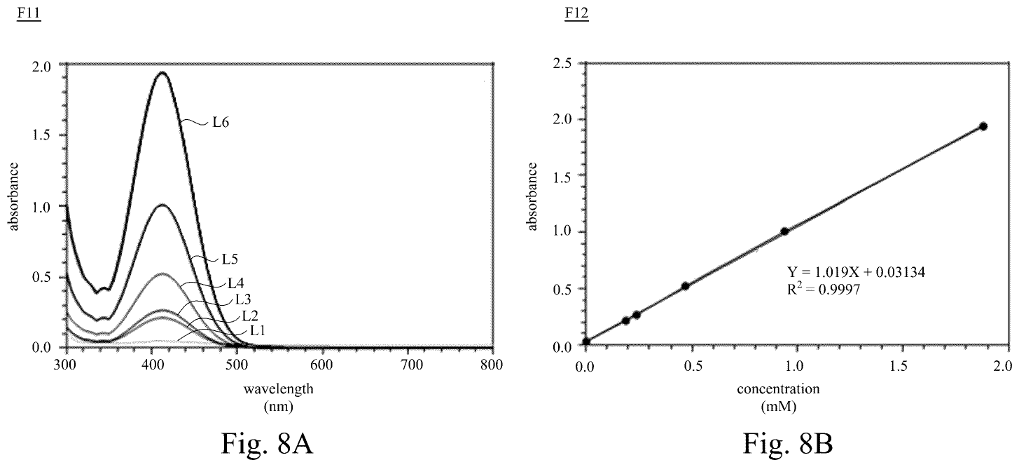

In this experiment, the modified glucose oxidase is prepared using the aforementioned method for [Preparation of Modified Redox Enzyme of the First Type](note: using the hydrophobic modification chain segment precursor of the first type), and the number of hydrophobic modification chain segments on the modified glucose oxidase is determined using a UV-Vis spectrophotometer. Specifically, the structure of Formula (C) in the hydrophobic modification chain segment precursor of the first type is dissolved in dimethyl sulfoxide (DMSO) at various concentrations, and the absorbance at a wavelength of 410 nm is measured (refer to FIG. 8A, which is a UV-Vis absorption spectrum (F11) of Formula (C) and the modified glucose oxidase at different concentrations in DMSO, where curves L2 to L6 represent concentrations of 0.188 mM, 0.24 mM, 0.47 mM, 0.94 mM, and 1.88 mM, respectively), from which a calibration curve is plotted (refer to FIG. 8B, which is a concentration-absorbance calibration curve (F12) for Formula (C)). Subsequently, the modified glucose oxidase at a concentration of 6.25 μM is dissolved in DMSO, and its absorbance at 410 nm is measured (refer to curve L1 in FIG. 8A). According to the calibration curve, the concentration of hydrophobic modification chain segments formed from Formula (C) in the modified glucose oxidase is calculated to be 21.56 μM, which is 3.45 times the concentration of the modified glucose oxidase, indicating that the number of hydrophobic modification chain segments per modified glucose oxidase molecule is approximately three.

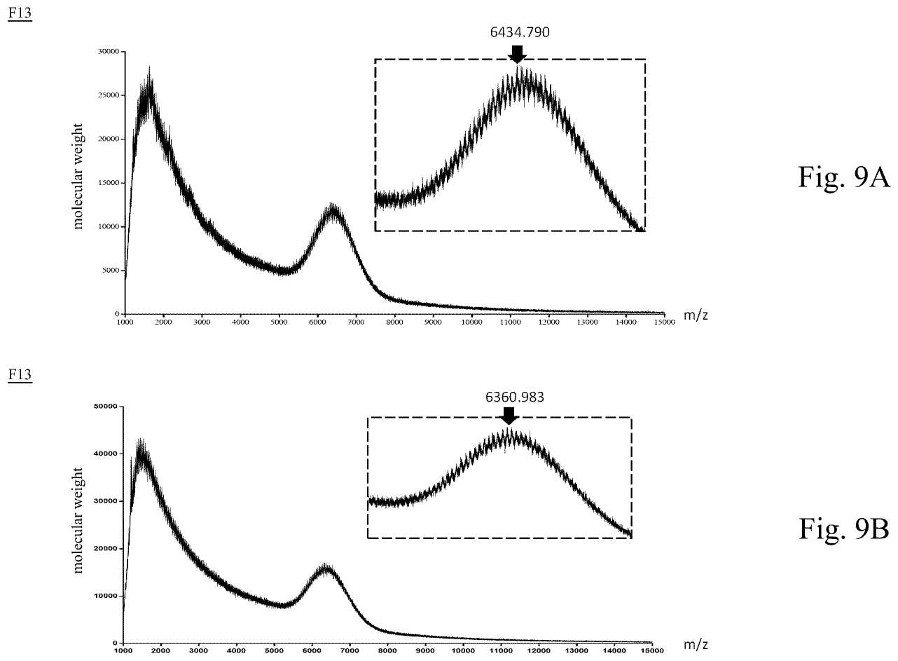

Furthermore, the modified glucose oxidase and the unmodified glucose oxidase are analyzed using matrix-assisted laser desorption/ionization time-of-flight mass spectrometry (MALDI-TOF MS). The results are shown in FIG. 9A and FIG. 9B, which are the MALDI-MS spectra (F13) of the modified glucose oxidase and glucose oxidase, respectively. In FIG. 9A, the signal of the modified glucose oxidase appears at 6434.790 (where the molecular weight of the Formula (C) fragment is 530 g/mol), and in FIG. 9B, the signal of the glucose oxidase appears at 6360.983 (where each glucose oxidase molecule consists of two identical subunits, each with a molecular weight of approximately 74,200 Da), indicating that the average molar ratio of Formula (C) to glucose oxidase is approximately 1.6. That is, about 1.6 Formula (C) fragments are grafted onto each glucose oxidase molecule. Therefore, combining the results of UV-Vis analyses and MALDI-MS analyses, the coupling ratio of Formula (C) to glucose oxidase is approximately 1 to 3, indicating successful conjugation of the hydrophobic modification chain segments derived from Formula (C) with glucose oxidase.

### <Experiment 6: Stability of the Sensor>

In this experiment, a modified glucose oxidase is prepared using the aforementioned method for [Preparation of Modified Redox Enzyme of the First Type](note: using the hydrophobic modification chain segment precursor of the first type), and is further processed into a nanoparticle composition using the aforementioned method for [Preparation of Nanoparticle Composition Using Modified Redox Enzyme of the First Type]. This nanoparticle composition and the modified glucose oxidase are respectively fabricated into sensor E1 and sensor E2. The layered structure of sensor E1 is as follows: platinum electrode/poly(3,4-ethylenedioxythiophene) (PEDOT) layer/composite of modified glucose oxidase, bovine serum albumin, and glutaraldehyde/Nafion™ film. The layered structure of sensor E2 is the same as the layered structure of sensor E1, except that the composite of modified glucose oxidase, bovine serum albumin, and glutaraldehyde in sensor E1 is replaced with modified glucose oxidase. Subsequently, stability tests are conducted on sensor E1 and sensor E2.

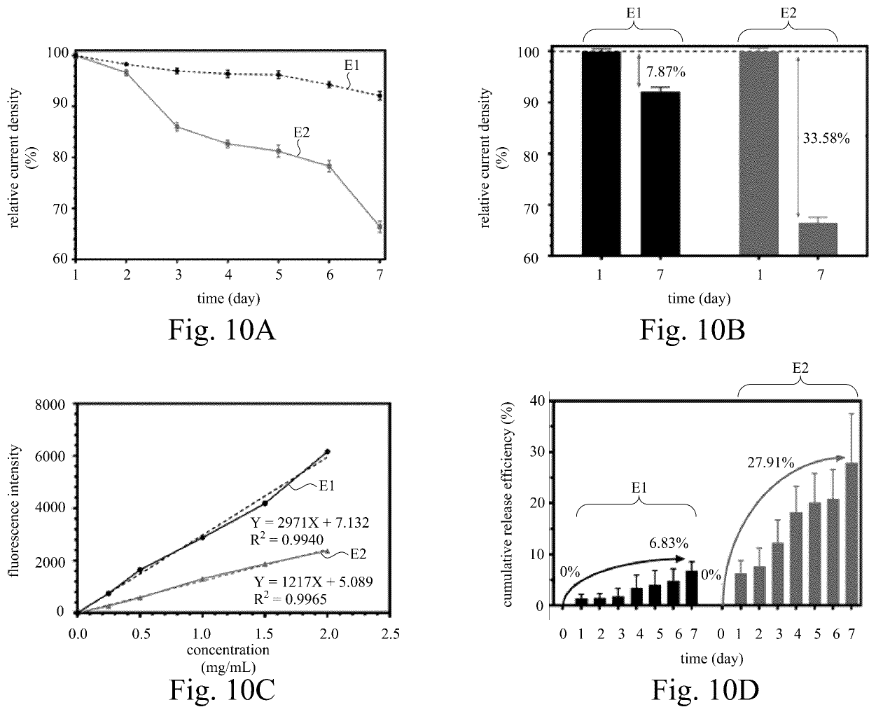

The results are shown in FIGS. 10A to 10D. FIG. 10A is a graph showing the change in current density of sensors E1 and E2 over a 7-day period in phosphate buffer solution. FIG. 10B is a bar chart showing the change in current density of sensors E1 and E2 after 7 days in phosphate buffer solution. The results indicate that sensor E1 exhibits significantly lower activity loss. FIG. 10C is a fluorescence intensity-concentration plot of sensors E1 and E2 in phosphate buffer solution, showing that the modified glucose oxidase in sensor E1 has lower solubility than the glucose oxidase in sensor E2, and thus demonstrates greater stability. FIG. 10D is a chart showing the cumulative release efficiency of sensors E1 and E2 after 7 days in phosphate buffer solution. The results show that the modified glucose oxidase in sensor E1, due to its nanostructure, possesses enhanced stability and is less likely to leach out in large quantities over a short period, which effectively reduces the risk of enzyme deactivation. This contributes to sustained enzyme activity over extended periods and improves the reliability and accuracy of the sensor during long-term operation.

### <Experiment 7: Specificity of Modified Redox Enzyme>

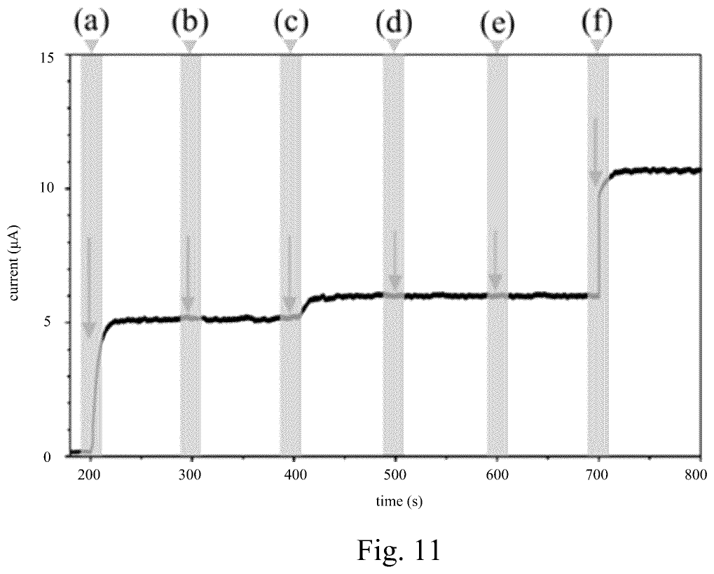

In this experiment, specificity testing of the aforementioned sensor E1 is conducted. Please refer to FIG. 11, which shows the current response curves of sensor E1 upon the addition of different substrates (applied voltage: 0.65 V). Specifically, substrates are sequentially added at time points (a) to (f): glucose (2 mM), ascorbic acid (0.34 mM), acetaminophen (1.3 mM), fructose (2 mM), galactose (3.3 mM), and glucose (2 mM). The results show a significant increase in current signal upon the addition of glucose, indicating that the sensor exhibits good selectivity and specificity for glucose. In contrast, no significant current response is observed upon the addition of common interfering substances such as ascorbic acid, acetaminophen, fructose, and galactose, demonstrating that the sensor possesses high selectivity and effectively avoids nonspecific signal interference, thereby enhancing the reliability and accuracy of glucose detection in actual biological fluids.

### <Experiment 8: Analysis of the Effect of Temperature on the Stability of Enzyme Nanoparticles>

In this experiment, a modified glucose oxidase is prepared using the method described previously under [Preparation of Modified Redox Enzyme of the First Type](note: using the first hydrophobic modification chain segment precursor), and the modified glucose oxidase is further processed into a nanoparticle composition using the method described under [Preparation of Nanoparticle Composition Using Modified Redox Enzyme of the First Type]. Subsequently, an enzyme reaction solution containing the nanoparticle composition (concentration: 0.1 μg/mL), HRP (concentration: 0.4 μg/mL), and ABTS (concentration: 2.5 mM) is prepared. A volume of 400 μL of the reaction solution is transferred to a J-815 circular dichroism spectrometer for subsequent measurements, and a circulating constant-temperature water bath is activated to regulate the temperature to the desired setting. Once the reaction system stabilizes at the target temperature, the detection wavelength is set to 740 nm (the characteristic absorption wavelength of ABTS⁺·), and glucose is added as the reaction substrate. The change in absorbance over time is then recorded. Using the standard calibration curve of ABTS⁺· and the rate of absorbance change, the corresponding concentration change of ABTS⁺· is calculated. Based on the Michaelis-Menten kinetic model, various enzyme kinetic parameters are derived to evaluate the catalytic efficiency and stability of the nanoparticle composition under different temperature conditions.

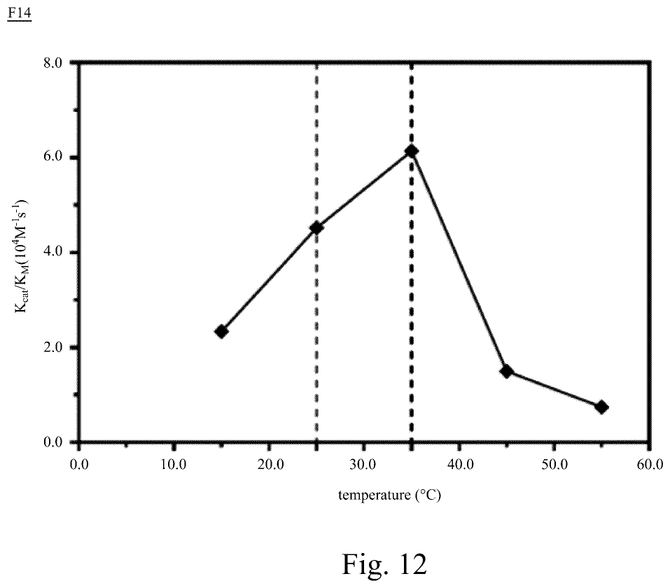

The experimental results are shown in FIG. 12, which presents the reaction rate curves of the nanoparticle composition at different temperatures (graph (F14)). FIG. 12 shows that the nanoparticle composition exhibits enzymatic activity within the temperature range of 15° C. to 60° C., with the highest catalytic efficiency observed at 35° C., where K_{cat}/K_{M} is approximately 6.0×10⁴ s⁻¹·M⁻¹. Additionally, it maintains high reaction activity at room temperature (25° C.), indicating that the nanoparticle composition has good stability and catalytic capability within commonly used operating temperature ranges. Overall, under conditions from 15° C. to 60° C., the catalytic efficiency of the nanoparticle composition toward glucose ranges between greater than 0.5×10⁴ s⁻¹·M⁻¹ and less than 6.5×10⁴ s⁻¹·M⁻¹ (for example, 10⁴ s⁻¹·M⁻¹, 1.5×10⁴ s⁻¹·M⁻¹, 2×10⁴ s⁻¹·M⁻¹, 2.5×10⁴ s⁻¹·M⁻¹, 3×10⁴ s⁻¹·M⁻¹, 3.5×10⁴ s⁻¹·M⁻¹, 4×10⁴ s⁻¹·M⁻¹, 4.5×10⁴ s⁻¹·M⁻¹, 5.5×10⁴ s⁻¹·M⁻¹, 6×10⁴ s⁻¹·M⁻¹), demonstrating that the nanoparticle composition possesses good temperature tolerance and broad application flexibility.

Although the present disclosure has been described with reference to the above embodiments, it is not intended to limit the disclosure. Those skilled in the art may make various modifications and refinements without departing from the spirit and scope of the disclosure. Therefore, the protection scope of the present disclosure shall be defined by the appended claims.

What is claimed is:

1. A modified oxidoreductase, comprising:
a dehydrogenated oxidoreductase having at least one dehydrogenated thiol group or at least one dehydrogenated amino group; and
a modification chain segment, wherein:
when the dehydrogenated oxidoreductase has the at least one dehydrogenated thiol group, the modification chain segment comprises a structure represented by Formula (1) and is bonded to the at least one dehydrogenated thiol group: 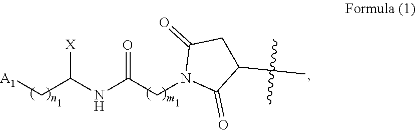
wherein A₁ is a first chain segment having a first π-conjugated system, X is hydrogen, alkyl, carboxyl, amide, or ester group, n₁ is an integer from 0 to 20, and m₁ is an integer from 0 to 20;
when the dehydrogenated oxidoreductase has the at least one dehydrogenated amino group, the modification chain segment comprises a structure represented by Formula (2) and is bonded to the at least one dehydrogenated amino group: 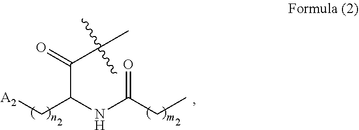
wherein A₂ is a second chain segment having a second π-conjugated system, n₂ is an integer from 0 to 20, and m₂ is an integer from 0 to 20.

2. The modified oxidoreductase of claim 1, wherein the first π-conjugated system or the second π-conjugated system is a π-conjugated system within an aromatic ring.

3. The modified oxidoreductase of claim 1, wherein the first chain segment and the second chain segment are each represented by Formula (3), Formula (4), or Formula (5):

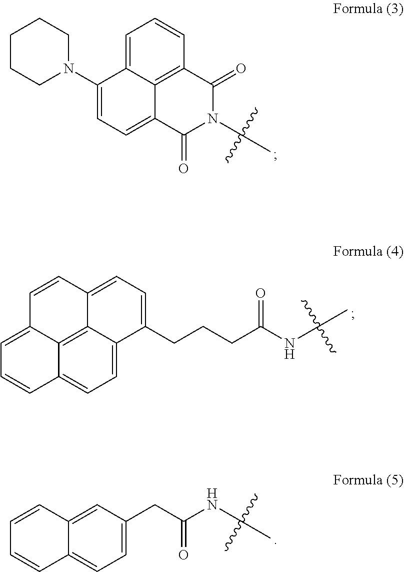 [SMILES: *N1C(=O)c2cccc3c(N4CCCCC4)ccc(c23)C1=O ; *NC(=O)CCCc1ccc2ccc3cccc4ccc1c2c34 ; *NC(=O)Cc1ccc2ccccc2c1]

4. The modified oxidoreductase of claim 1, wherein the dehydrogenated oxidoreductase is obtained by dehydrogenation of glucose oxidase, glucose dehydrogenase, pyruvate oxidase, xanthine oxidase, acetylcholinesterase, lactate oxidase, urate oxidase, uricase, pyrroloquinoline quinone-dependent glucose dehydrogenase-A, pyrroloquinoline quinone-dependent glucose dehydrogenase-B, NAD(P)-dependent glutamate dehydrogenase, FAD-dependent glutamate dehydrogenase, cholesterol oxidase, or a sulfur-containing enzyme.

5. The modified oxidoreductase of claim 1, wherein a number of the at least one dehydrogenated thiol group is from 1 to 20, or a number of the at least one dehydrogenated amino group is from 1 to 20.

6. A preparation method of a nanoparticle composition, comprising:
placing a plurality of the modified oxidoreductases of claim 1 in an aldehyde or aldonic acid solution at a concentration of 0.1 mM to 0.31 mM.

7. The preparation method of claim 6, wherein the aldehyde or aldonic acid solution is a monoaldehyde solution, dialdehyde solution, aromatic aldehyde solution, α,β-unsaturated aldehyde solution, hydroxyaldehyde solution, ketoaldehyde solution, aldonic acid solution, or combinations thereof.

8. A nanoparticle composition, comprising:
a plurality of enzyme nanoparticles, each of the enzyme nanoparticles being formed by crosslinking a plurality of modified oxidoreductases and a plurality of aldehyde or aldonic acid molecules, wherein each of the modified oxidoreductases comprises:
a dehydrogenated oxidoreductase having at least one dehydrogenated thiol group or at least one dehydrogenated amino group; and
a modification chain segment, wherein:
when the dehydrogenated oxidoreductase has the at least one dehydrogenated thiol group, the modification chain segment comprises a structure represented by Formula (1) and is bonded to the at least one dehydrogenated thiol group: 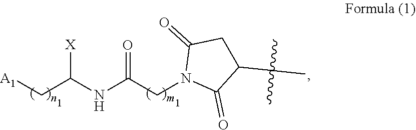
wherein A₁ is a first chain segment having a first π-conjugated system, X is hydrogen, alkyl, carboxyl, amide, or ester group, n₁ is an integer from 0 to 20, and m₁ is an integer from 0 to 20;
when the dehydrogenated oxidoreductase has the at least one dehydrogenated amino group, the modification chain segment comprises a structure represented by Formula (2) and is bonded to the at least one dehydrogenated amino group: 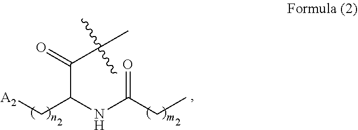
wherein A₂ is a second chain segment having a second π-conjugated system, n₂ is an integer from 0 to 20, and m₂ is an integer from 0 to 20.

9. The nanoparticle composition of claim 8, wherein an average diameter of the enzyme nanoparticles is from 10 nanometers to 5000 nanometers.

10. The nanoparticle composition of claim 8, wherein the aldehyde or aldonic acid molecules are monoaldehyde, dialdehyde, aromatic aldehyde, α,β-unsaturated aldehyde, hydroxyaldehyde, ketoaldehyde, aldonic acid, or combinations thereof.

11. The nanoparticle composition of claim 8, wherein the dehydrogenated oxidoreductase is obtained by dehydrogenation of glucose oxidase, and the nanoparticle composition has a catalytic efficiency (K_{cat}/K_{M}) for glucose greater than 0.5×10⁴ s⁻¹·M⁻¹ and less than 5.0×10⁴ s⁻¹·M⁻¹, wherein K_{cat} is the catalytic constant and K_{M} is the Michaelis constant.

12. An electrical signal sensor, comprising:
an electrode layer; and
a sensing layer disposed on a surface of the electrode layer, wherein the sensing layer comprises the nanoparticle composition of claim 8.

13. A nanomedicine, comprising:
the nanoparticle composition of claim 8.

14. A nanomedicine complex, comprising:
the nanoparticle composition of claim 8; and
a drug molecule bound in a releasable form to any of the modified oxidoreductases in the nanoparticle composition.

15. The nanomedicine complex of claim 14, wherein the drug molecule is an anticancer drug.
